SEQUENCE LISTING (65 sequences)

SEQ ID NO: 1 - protein, 115 aa, synthetic construct
EVQLVQSGPEVKKPGATVKISCKTSGYTFTEYTIHWVRQAPGKGLEWIGNINPREGGTTYNQKFEDKATLTVDKSTDTAYMELSSLRSEDTAVYYCAAGWNFDSWGQGTLLTVSS

SEQ ID NO: 2 - protein, 107 aa, synthetic construct
DIQMTQSPSSLSTSVGDRVTLTCKASQDIGTAVDWYQQKPGTSPKLLIYWASTRHTGIPSRFSGSGSGTDFTLTISSLQPEDAADYYCQQYSSYPLTFGPGTKVEIK

SEQ ID NO: 3 - protein, 5 aa, synthetic construct
EYTIH

SEQ ID NO: 4 - protein, 7 aa, synthetic construct
GYTFTEY

SEQ ID NO: 5 - protein, 10 aa, synthetic construct
GYTFTEYTIH

SEQ ID NO: 6 - protein, 17 aa, synthetic construct
NINPREGGTTYNQKFED

SEQ ID NO: 7 - protein, 6 aa, synthetic construct
NPREGG

SEQ ID NO: 8 - protein, 8 aa, synthetic construct
INPREGGT

SEQ ID NO: 9 - protein, 10 aa, synthetic construct
NINPREGGTT

SEQ ID NO: 10 - protein, 6 aa, synthetic construct
GWNFDS

SEQ ID NO: 11 - protein, 8 aa, synthetic construct
AAGWNFDS

SEQ ID NO: 12 - protein, 11 aa, synthetic construct
KASQDIGTAVD

SEQ ID NO: 13 - protein, 6 aa, synthetic construct
QDIGTA

SEQ ID NO: 14 - protein, 7 aa, synthetic construct
WASTRHT

SEQ ID NO: 15 - skipped

SEQ ID NO: 16 - protein, 9 aa, synthetic construct
QQYSSYPLT

SEQ ID NO: 17 - protein, 444 aa, synthetic construct
EVQLVQSGPEVKKPGATVKISCKTSGYTFTEYTIHWVRQAPGKGLEWIGNINPREGGTTYNQKFEDKATLTVDKSTDTAYMELSSLRSEDTAVYYCAAGWNFDSWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 214 aa, synthetic construct
DIQMTQSPSSLSTSVGDRVTLTCKASQDIGTAVDWYQQKPGTSPKLLIYWASTRHTGIPSRFSGSGSGTDFTLTISSLQPEDAADYYCQQYSSYPLTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 107 aa, synthetic construct [sequence omitted]

SEQ ID NO: 21 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 22 - protein, 707 aa, Homo sapiens
KSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 23 - protein, 707 aa, Macaca fascicularis
KSSSEATNITPKHNMKAFLDELKAENIKKFLHNFTQIPHLAGTEQNFQLAKQIQSQWKEFGLDSVELTHYDVLLSYPNKTHPNYISIINEDGNEIFNTSLFEPPPAGYENVSDIVPPFSAFSPQGMPEGDLVYVNYARTEDFFKLERDMKINCSGKIVIARYGKVFRGNKVKNAQLAGATGVILYSDPADYFAPGVKSYPDGWNLPGGGVQRGNILNLNGAGDPLTPGYPANEYAYRRGIAEAVGLPSIPVHPIGYYDAQKLLEKMGGSASPDSSWRGSLKVPYNVGPGFTGNFSTQKVKMHIHSTSEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVYNLTKELESPDEGFEGKSLYESWTKKSPSPEFSGMPRISKLGSGNDFEVFFQRLGIASGRARYTKNWETNKFSSYPLYHSVYETYELVEKFYDPMFKYHLTVAQVRGGMVFELANSVVLPFDCRDYAVVLRKYADKIYNISMKHPQEMKTYSVSFDSLFSAVKNFTEIASKFSERLRDFDKSNPILLRMMNDQLMFLERAFIDPLGLPDRPFYRHVIYAPSSHNKYAGESFPGIYDALFDIESKVDPSQAWGEVKRQISIATFTVQAAAETLSEVA

SEQ ID NO: 24 - protein, 708 aa, unidentified
  REGION 1..708: note = Canine PSMA
KSPSEPTNIVPQHDMKKAFLDELKAENIKRFLYNFTRLPHLAGTERNFQLAKQIQSQWKEFGLDSVELAHYDVLLSYPNKTHPNYISIIDEDGNEIFNSSLFEPPPPGYENVSDVVPPFNAFSPQGMPEGDLVYVNYAQTEDFFKLERDLKINCSGKIVIARYGKIFRGNKVKNAQLAGAKGVILYSDPADYFAPGVKSYPDGWNLPGGGVQRGNILNLNGAGDPLTPGYPANEYAYRREVTEAVGLPSIPVHPIGYNDAQKLLEKMGGAAPPDSSWRGGLQVPYNVGPGFTGNSSTQKVKMHIHSNNKVTRIYNVIGTLRGAVEPDRYVILGGHRDSWVFGGIDPQSGAAVVHEIVRSFGTLKKEGWRPRRTILFASWDAEEFGLLGSTEWAEENSRVLQERGVAYINADSSIEGNYTLRVDCTPLMYSLVYNLTKELQSPDEGFEGKSLYESWNEKSPSPEFSGMPRISKLGSGNDFEVFFQRLGISSGRARYTKNWETSKFRSYPLYHSVYETYELVEKFYDPTFKYHLTVAQVRGGMVFELANSIVLPFDCRDYAVVLRKYADKIYNISMKHPQEMKTYSVSFDSLFSAVKNFTEIASKFNERLEDLDKKNPILLRIMNDQLMFLERAFIDPLGLPDRPFYRHIIYAPSSHNKYAGESFPGIYDALFDIESKVDPSKAWGEVKRQISIAAFTVQAAAGTLREVA

SEQ ID NO: 25 - protein, 708 aa, unidentified
  REGION 1..708: note = Rat PSMA
KPSNDSTSSVSYPGMKKAFLQELKAENIKKFLYNFTRTPHLAGTQHNFELAKQIHAQWKEFGLDLVELSDYDVLLSYPNKTHPNYISIINEDGNEIFKTSLAELSPPGYENISDVVPPYSAFSPQGTPEGDLVYVNYARTEDFFKLERVMKINCSGKIVIARYGQVFRGNKVKNAQLAGAKGIILYSDPADYFVPGVKSYPDGWNLPGGGVQRGNVLNLNGAGDPLTPGYPANEYAYRHEFTEAVGLPSIPVHPIGYDDAQKLLEHMGGSAPPDSSWKGGLKVPYNVGPGFAGNFSKQKVKLHIHSYNKVTRIYNVIGTLKGAVEPDRYVILGGHRDAWVFGGIDPQSGAAVVHEIVRTFGTLKKKGWRPRRTILFASWDAEEFGLLGSTEWAEEHSRLLQERGVAYINADSSIEGNYTLRVDCTPLMHSLVYNLTKELPSPDEGFEGKSLYDSWKEKSPSTEFIGMPRISKLGSGNDFEVFFQRLGIASGRARYTKNWKNNKVSSYPLYHSVYETYELVEKFYDPTFKYHLTVAQVRGAMVFELANSIVLPFDCQSYAVALKKHAETIYNISMNHPQEMKAYMISFDSLFSAVNNFTDVASKFNQRLQDLDKSNPILLRILNDQLMYLERAFIDPLGLPGRPFYRHIIYAPSSHNKYAGESFPGIYDALFDINNKVDTSKAWREVKRQISIAAFTVQAAAETLREVD

SEQ ID NO: 26 - protein, 709 aa, Mus musculus
IKPSNEATGNVSHSGMKKEFLHELKAENIKKFLYNFTRTPHLAGTQNNFELAKQIHDQWKEFGLDLVELSHYDVLLSYPNKTHPNYISIINEDGNEIFKTSLSEQPPPGYENISDVVPPYSAFSPQGTPEGDLVYVNYARTEDFFKLEREMKISCSGKIVIARYGKVFRGNMVKNAQLAGAKGMILYSDPADYFVPAVKSYPDGWNLPGGGVQRGNVLNLNGAGDPLTPGYPANEHAYRHELTNAVGLPSIPVHPIGYDDAQKLLEHMGGPAPPDSSWKGGLKVPYNVGPGFAGNFSTQKVKMHIHSYTKVTRIYNVIGTLKGALEPDRYVILGGHRDAWVFGGIDPQSGAAVVHEIVRSFGTLKKKGRRPRRTILFASWDAEEFGLLGSTEWAEEHSRLLQERGVAYINADSSIEGNYTLRVDCTPLMYSLVYNLTKELQSPDEGFEGKSLYDSWKEKSPSPEFIGMPRISKLGSGNDFEVFFQRLGIASGRARYTKNWKTNKVSSYPLYHSVYETYELVVKFYDPTFKYHLTVAQVRGAMVFELANSIVLPFDCQSYAVALKKYADTIYNISMKHPQEMKAYMISFDSLFSAVNNFTDVASKFNQRLQELDKSNPILLRIMNDQLMYLERAFIDPLGLPGRPFYRHIIYAPSSHNKYAGESFPGIYDALFDISSKVNASKAWNEVKRQISIATFTVQAAAETLREVA

SEQ ID NO: 27 - protein, 115 aa, synthetic construct
EVQLVQSGPEVKKPGATVKISCKTSGYTFTTYTIHWVRQAPGKGLEWIGNINPETGGTTYNQKFEDKATLTVDKSTDTAYMELSSLRSEDTAVYYCAAGWNFDYWGQGTLLTVSS

SEQ ID NO: 28 - protein, 107 aa, synthetic construct
DIQMTQSPSSLSTSVGDRVTLTCKASQDIGTAVDWYQQKPGKSPKLLIYWASTTHEGIPSRFSGSGSGTDFTLTISSLQPEDAADYYCQQYSSYPLTFGPGTKVEIK

SEQ ID NO: 29 - protein, 5 aa, synthetic construct
TYTIH

SEQ ID NO: 30 - protein, 7 aa, synthetic construct
GYTFTTY

SEQ ID NO: 31 - protein, 8 aa, synthetic construct
GYTFTTYT

SEQ ID NO: 32 - protein, 10 aa, synthetic construct
GYTFTTYTIH

SEQ ID NO: 33 - protein, 17 aa, synthetic construct
NINPETGGTTYNQKFED

SEQ ID NO: 34 - protein, 6 aa, synthetic construct
NPETGG

SEQ ID NO: 35 - protein, 8 aa, synthetic construct
INPETGGT

SEQ ID NO: 36 - protein, 10 aa, synthetic construct
NINPETGGTT

SEQ ID NO: 37 - protein, 6 aa, synthetic construct
GWNFDY

SEQ ID NO: 38 - protein, 8 aa, synthetic construct
AAGWNFDY

SEQ ID NO: 39 - protein, 11 aa, synthetic construct
KASQDIGTAVD

SEQ ID NO: 40 - protein, 6 aa, synthetic construct
QDIGTA

SEQ ID NO: 41 - protein, 7 aa, synthetic construct
WASTTHE

SEQ ID NO: 42 - skipped

SEQ ID NO: 43 - protein, 9 aa, synthetic construct
QQYSSYPLT

SEQ ID NO: 44 - protein, 444 aa, synthetic construct
EVQLVQSGPEVKKPGATVKISCKTSGYTFTTYTIHWVRQAPGKGLEWIGNINPETGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 214 aa, synthetic construct
DIQMTQSPSSLSTSVGDRVTLTCKASQDIGTAVDWYQQKPGKSPKLLIYWASTTHEGIPSRFSGSGSGTDFTLTISSLQPEDAADYYCQQYSSYPLTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 115 aa, synthetic construct
EVQLVQSGPEVKKPGATVKISCKTSGYTFTTYTIHWVRQAPGKGLEWIGNINPQTGGTTYNQKFEDKATLTVDKSTDTAYMELSSLRSEDTAVYYCAAGWNFDYWGQGTLLTVSS

SEQ ID NO: 47 - protein, 107 aa, synthetic construct
DIQMTQSPSSLSTSVGDRVTLTCKASQDIGTAVDWYQQKPGKSPKLLIYWASTTHEGIPSRFSGSGSGTDFTLTISSLQPEDAADYYCQQYSQYPLTFGPGTKVEIK

SEQ ID NO: 48 - protein, 5 aa, synthetic construct
TYTIH

SEQ ID NO: 49 - protein, 17 aa, synthetic construct
NINPQTGGTTYNQKFED

SEQ ID NO: 50 - protein, 6 aa, synthetic construct
GWNFDY

SEQ ID NO: 51 - protein, 11 aa, synthetic construct
KASQDIGTAVD

SEQ ID NO: 52 - protein, 7 aa, synthetic construct
WASTTHE

SEQ ID NO: 53 - protein, 9 aa, synthetic construct
QQYSQYPLT

SEQ ID NO: 54 - protein, 444 aa, synthetic construct
EVQLVQSGPEVKKPGATVKISCKTSGYTFTTYTIHWVRQAPGKGLEWIGNINPQTGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 214 aa, synthetic construct
DIQMTQSPSSLSTSVGDRVTLTCKASQDIGTAVDWYQQKPGKSPKLLIYWASTTHEGIPSRFSGSGSGTDFTLTISSLQPEDAADYYCQQYSQYPLTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 115 aa, synthetic construct
EVQLVQSGPEVKKPGATVKISCKTSGYTFTEYTIHWVRQAPGKGLEWIGNINPREGGTTYNQKFEDKATLTVDKSTDTAYMELSSLRSEDTAVYYCAAGWNFDYWGQGTLLTVSS

SEQ ID NO: 57 - protein, 107 aa, synthetic construct
DIQMTQSPSSLSTSVGDRVTLTCKASQDIGTAVDWYQQKPGKSPKLLIYWASTRHEGIPSRFSGSGSGTDFTLTISSLQPEDAADYYCQQYSSYPLTFGPGTKVEIK

SEQ ID NO: 58 - protein, 5 aa, synthetic construct
EYTIH

SEQ ID NO: 59 - protein, 17 aa, synthetic construct
NINPREGGTTYNQKFED

SEQ ID NO: 60 - protein, 6 aa, synthetic construct
GWNFDY

SEQ ID NO: 61 - protein, 11 aa, synthetic construct
KASQDIGTAVD

SEQ ID NO: 62 - protein, 7 aa, synthetic construct
WASTRHE

SEQ ID NO: 63 - protein, 9 aa, synthetic construct
QQYSSYPLT

SEQ ID NO: 64 - protein, 444 aa, synthetic construct
EVQLVQSGPEVKKPGATVKISCKTSGYTFTEYTIHWVRQAPGKGLEWIGNINPREGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 214 aa, synthetic construct
DIQMTQSPSSLSTSVGDRVTLTCKASQDIGTAVDWYQQKPGKSPKLLIYWASTRHEGIPSRFSGSGSGTDFTLTISSLQPEDAADYYCQQYSSYPLTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0308273 A1
(43) Pub. Date: Oct. 8, 2026

(54) ANTI-PSMA ANTIBODIES AND USES THEREOF

(71) Applicant: Bristol-Myers Squibb Company, Princeton, NJ (US)

(72) Inventors: Robert James Hoey, Santee, CA (US); Joshua Carl Goldstein, San Diego, CA (US)

(73) Assignee: Bristol-Myers Squibb Company, Princeton, NJ (US)

(21) Appl. No.: 19/636,107

(22) Filed: Apr. 1, 2026

Related U.S. Application Data
(60) Provisional application No. 63/783,497, filed on Apr. 4, 2025.

(51) Int. Cl. C07K 16/30; A61K 39/00; A61K 47/68; A61P 35/00; C12N 15/63

(52) U.S. Cl. CPC C07K 16/3069; A61K 47/6817; A61K 47/6869; A61P 35/00; C12N 15/63; A61K 2039/505; C07K 2317/24; C07K 2317/31; C07K 2317/40; C07K 2317/565; C07K 2317/92

(57) ABSTRACT

The present application relates to particular anti-PSMA antibodies, nucleic acids encoding the antibodies, vectors and host cells comprising the nucleic acids, and methods of making and using the antibodies, as well as immunoconjugates comprising the anti-PSMA antibodies.

### CROSS-REFERENCE TO RELATED APPLICATIONS

This application claims the benefit of priority to U.S. Provisional Application No. 63/783,497, filed Apr. 4, 2025, the contents of which are incorporated by reference herein in its entirety.

### INCORPORATION BY REFERENCE OF SEQUENCE LISTING

The application contains a Sequence Listing which has been submitted electronically in .XML format and is hereby incorporated by reference in its entirety. Said .XML copy, created on Mar. 30, 2026, is named “01277-0097-00US.xml” and is 64,502 bytes in size. The sequence listing contained in this .XML file is part of the specification and is hereby incorporated by reference herein in its entirety.

### FIELD

The present application relates to anti-PSMA antibodies, nucleic acids encoding the antibodies, vectors and host cells comprising the nucleic acids, and methods of making and using the antibodies.

### BACKGROUND

There exists a need for safe and effective agents and methods for treating, preventing and managing cancer, including for cancers that are refractory to standard treatments, while reducing or avoiding the toxicities and/or side effects associated with some existing therapies.

### SUMMARY

The inventors have found anti-PSMA antibodies with superior binding, selectivity, species cross-reactivity, developability, immunogenicity, pharmacokinetics, and in vivo biodistribution, as compared to a benchmark antibody. The present disclosure relates to anti-PSMA antibodies, nucleic acids encoding the antibodies, vectors and host cells comprising the nucleic acids, and methods of both making and using the antibodies. For example, embodiments of the disclosure include the following:

Embodiment 1. An isolated antibody or antigen-binding portion thereof that specifically binds to PSMA, wherein the antibody or antigen-binding portion thereof comprises a heavy chain complementarity determining region 1 (HCDR1) comprising an amino acid sequence selected from SEQ ID NOs: 3-5; a heavy chain complementarity determining region 2 (HCDR2) comprising an amino acid sequence selected from SEQ ID NOs: 6-9; a heavy chain complementarity determining region 3 (HCDR3) comprising the amino acid sequence of SEQ ID NO: 10 or 11; a light chain complementarity determining region 1 (LCDR1) comprising the amino acid sequence of SEQ ID NO: 12 or 13; a light chain complementarity determining region 2 (LCDR2) comprising the amino acid sequence of SEQ ID NO: 14 or 15; and a light chain complementarity determining region 3 (LCDR3) comprising the amino acid sequence of SEQ ID NO: 16.

Embodiment 2. The isolated antibody or antigen-binding portion thereof of embodiment 1, wherein the antibody or antigen-binding portion thereof comprises:
a) HCDR1 comprising the amino acid sequence of SEQ ID NO: 3, HCDR2 comprising the amino acid sequence of SEQ ID NO: 6, HCDR3 comprising the amino acid sequence of SEQ ID NO: 10, LCDR1 comprising the amino acid sequence of SEQ ID NO: 12, LCDR2 comprising the amino acid sequence of SEQ ID NO: 14, LCDR3 comprising the amino acid sequence of SEQ ID NO: 16;
b) HCDR1 comprising the amino acid sequence of SEQ ID NO: 4, HCDR2 comprising the amino acid sequence of SEQ ID NO: 7, HCDR3 comprising the amino acid sequence of SEQ ID NO: 10, LCDR1 comprising the amino acid sequence of SEQ ID NO: 12, LCDR2 comprising the amino acid sequence of SEQ ID NO: 14, LCDR3 comprising the amino acid sequence of SEQ ID NO: 16;
c) HCDR1 comprising the amino acid sequence of SEQ ID NO: 4, HCDR2 comprising the amino acid sequence of SEQ ID NO: 8, HCDR3 comprising the amino acid sequence of SEQ ID NO: 11, LCDR1 comprising the amino acid sequence of SEQ ID NO: 13, LCDR2 comprising the amino acid sequence of SEQ ID NO: 15, LCDR3 comprising the amino acid sequence of SEQ ID NO: 16; or
d) HCDR1 comprising the amino acid sequence of SEQ ID NO: 5, HCDR2 comprising the amino acid sequence of SEQ ID NO: 9, HCDR3 comprising the amino acid sequence of SEQ ID NO: 10, LCDR1 comprising the amino acid sequence of SEQ ID NO: 12, LCDR2 comprising the amino acid sequence of SEQ ID NO: 14, LCDR3 comprising the amino acid sequence of SEQ ID NO: 16.

Embodiment 3. The isolated antibody or antigen-binding portion thereof of embodiment 1 or embodiment 2, wherein the antibody or antigen-binding portion thereof comprises a heavy chain variable region (VH) comprising an amino acid sequence that is at least 90%, 91%, 92%, 93%, 94%, 95%, 96%, 97%, 98%, or 99% identical to the amino acid sequence of SEQ ID NO: 1, and a light chain variable region (VL) comprising an amino acid sequence that is at least 90%, 91%, 92%, 93%, 94%, 95%, 96%, 97%, 98%, or 99% identical to the amino acid sequence of SEQ ID NO: 2; optionally wherein the glutamic acid (E) at the N-terminus of SEQ ID NO: 1 may be replaced with a pyroglutamic acid (pE).

Embodiment 4. The isolated antibody or antigen-binding portion thereof of any one of embodiments 1-3, wherein the antibody or antigen-binding portion thereof comprises a heavy chain variable region (VH) comprising the amino acid sequence of SEQ ID NO: 1, and a light chain variable region (VL) comprising the amino acid sequence selected of SEQ ID NO: 2; optionally wherein the glutamic acid (E) at the N-terminus of SEQ ID NO: 1 may be replaced with a pyroglutamic acid (pE).

Embodiment 5. The isolated antibody or antigen-binding portion thereof of any one of embodiments 1-4, wherein the antibody or antigen-binding portion thereof is a monoclonal antibody.

Embodiment 6. The isolated antibody or antigen-binding portion thereof of any one of embodiments 1-5, wherein the antibody or antigen-binding portion thereof is a human antibody, chimeric antibody, or a humanized antibody.

Embodiment 7. The isolated antibody or antigen-binding portion thereof of any one of embodiments 1-6, wherein the antibody or antigen-binding portion thereof is an IgG antibody.

Embodiment 8. The isolated antibody or antigen-binding portion thereof of embodiment 7, wherein the antibody is an IgG antibody lacking a C-terminal lysine in the heavy chain constant region.

Embodiment 9. The isolated antibody or antigen-binding portion thereof of embodiment 7 or embodiment 8, wherein the antibody is an IgG1 antibody, an IgG2 antibody, an IgG3 antibody, or an IgG4 antibody.

Embodiment 10. The isolated antibody or antigen-binding portion thereof of any one of embodiments 1-9, wherein the antibody is a full-length antibody.

Embodiment 11. The isolated antibody or antigen-binding portion thereof of any one of embodiments 1-10, wherein the antibody or antigen-binding portion thereof comprises a heavy chain (HC) comprising an amino acid sequence that is at least 90%, 91%, 92%, 93%, 94%, 95%, 96%, 97%, 98%, or 99% identical to the amino acid sequence of SEQ ID NO: 17, and a light chain (LC) comprising an amino acid sequence that is at least 90%, 91%, 92%, 93%, 94%, 95%, 96%, 97%, 98%, or 99% identical to the amino acid sequence of SEQ ID NO: 18; optionally wherein the glutamic acid (E) at the N-terminus of SEQ ID NO: 17 may be replaced with a pyroglutamic acid (pE).

Embodiment 12. The isolated antibody or antigen-binding portion thereof of any one of embodiments 1-10, wherein the antibody or antigen-binding portion thereof comprises a a heavy chain (HC) comprising the amino acid sequence of SEQ ID NO: 17, and a light chain (LC) comprising the amino acid sequence selected of SEQ ID NO: 18; optionally wherein the glutamic acid (E) at the N-terminus of SEQ ID NO: 17 may be replaced with a pyroglutamic acid (pE).

Embodiment 13. The isolated antibody or antigen-binding portion thereof of any one of embodiments 1-9, wherein the antibody or antigen-binding portion thereof is an antibody fragment selected from an Fv, single-chain Fv (scFv), Fab, Fab′, or (Fab′)₂.

Embodiment 14. The isolated antibody or antigen-binding portion thereof of any one of embodiments 1-13, wherein the antibody or antigen-binding portion thereof binds human PSMA.

Embodiment 15. The isolated antibody or antigen-binding portion thereof of embodiment 14, wherein human PSMA comprises the amino acid sequence of SEQ ID NO: 21 or SEQ ID NO: 26.

Embodiment 16. The isolated antibody or antigen-binding portion thereof of any one of embodiments 1-15, wherein the antibody or antigen-binding portion thereof binds to human PSMA with an affinity (K_{D}) of less than 10 nM or less than 5 nM.

Embodiment 17. The isolated antibody or antigen-binding portion thereof of any one of embodiments 1-15, wherein the antibody or antigen-binding portion thereof binds to cynomolgus monkey PSMA with an affinity (K_{D}) of less than 10 nM or less than 7 nM.

Embodiment 18. The isolated antibody or antigen-binding portion thereof of embodiment 16 or embodiment 17, wherein affinity is determined using surface plasmon resonance.

Embodiment 19. The isolated antibody or antigen-binding portion thereof of any one of embodiments 1-18, wherein the antibody or antigen-binding portion thereof is a bispecific antibody.

Embodiment 20. An antibody-drug conjugate comprising the antibody or antigen-binding portion thereof of any one of embodiments 1-19 and a cytotoxic agent.

Embodiment 21. A pharmaceutical composition comprising the isolated antibody or antigen-binding portion thereof of any one of embodiments 1-19 or the antibody-drug conjugate of embodiment 20 and a pharmaceutically acceptable carrier.

Embodiment 22. An isolated nucleic acid encoding the antibody or antigen-binding portion thereof of any one of embodiments 1-19.

Embodiment 23. A vector comprising the nucleic acid of embodiment 22.

Embodiment 24. A host cell comprising the nucleic acid of embodiment 22 or the vector of embodiment 23.

Embodiment 25. A host cell that expresses the antibody or antigen-binding portion thereof of any one of embodiments 1-19.

Embodiment 26. A method for making an anti-PSMA antibody or antigen-binding portion thereof, comprising culturing the host cell of embodiment 24 or 25 under conditions suitable for expression of the antibody or antigen-binding portion thereof.

Embodiment 27. The method of embodiment 26, further comprising recovering the antibody or antigen-binding portion thereof produced by the host cell.

Embodiment 28. A method of treating cancer in a subject in need thereof, comprising administering to the subject a therapeutically effective amount of the isolated antibody or antigen-binding portion thereof of any one of embodiments 1-19, the pharmaceutical composition of embodiment 21, or the antibody-drug conjugate of embodiment 20.

Embodiment 29. The method of embodiment 28, wherein the cancer is a PSMA+ cancer.

Embodiment 30. The method of embodiment 28 or embodiment 29, wherein the cancer is prostate cancer, castration resistant prostate cancer (CRPC), or metastatic castration resistant prostate cancer (mCRPC).

Embodiment 31. The method of any one of embodiments 28-30, wherein the subject is a human.

Embodiment 32. Use of the isolated antibody or antigen-binding portion thereof of any one of embodiments 1-19, the pharmaceutical composition of embodiment 21, or the antibody-drug conjugate of embodiment 20 for the preparation of a medicament for treating cancer in a subject in need thereof.

Embodiment 33. The use of embodiment 32, wherein the cancer is a PSMA+ cancer.

Embodiment 34. The use of embodiment 32 or embodiment 33, wherein the cancer is prostate cancer, castration resistant prostate cancer (CRPC), or metastatic castration resistant prostate cancer (mCRPC).

Embodiment 35. The use of any one of embodiments 32-24, wherein the subject is a human.

Embodiment 36. The isolated antibody or antigen-binding portion thereof of any one of embodiments 1-19, the pharmaceutical composition of embodiment 21, or the antibody-drug conjugate of embodiment 20 for use in treating cancer in a subject in need thereof.

Embodiment 37. The isolated antibody or antigen-binding portion thereof for use of embodiment 36, wherein the cancer is a PSMA+ cancer.

Embodiment 38. The isolated antibody or antigen-binding portion thereof for use of embodiment 36 or embodiment 37, wherein the cancer is prostate cancer, castration resistant prostate cancer (CRPC), or metastatic castration resistant prostate cancer (mCRPC).

Embodiment 39. The isolated antibody or antigen-binding portion thereof for use of any one of embodiments 36-38, wherein the subject is a human.

It is to be understood that both the foregoing general description and the following detailed description are exemplary and explanatory only and are not restrictive of the claims.

All references cited herein are incorporated in their entirety by reference.

### BRIEF DESCRIPTION OF THE FIGURES

FIG. 1 depicts numerous rounds of characterization and screening of rationally designed variant anti-PSMA antibodies.

### DETAILED DESCRIPTION OF CERTAIN EMBODIMENTS

### I. Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

Exemplary techniques used in connection with recombinant DNA, oligonucleotide synthesis, tissue culture and transformation (e.g., electroporation, lipofection), enzymatic reactions, and purification techniques are described, e.g., in Sambrook et al. Molecular Cloning: A Laboratory Manual (2nd ed., Cold Spring Harbor Laboratory Press, Cold Spring Harbor, N.Y. (1989)), among other places.

As used herein, the term “about” refers to a numeric value, including, for example, whole numbers, fractions, and percentages, whether or not explicitly indicated. The term “about” generally refers to a range of numerical values (e.g., +/−5-10% of the recited range) that one of ordinary skill in the art would consider equivalent to the recited value (e.g., having the same function or result). When terms such as “at least” and “about” precede a list of numerical values or ranges, the terms modify all of the values or ranges provided in the list. In some instances, the term about may include numerical values that are rounded to the nearest significant FIGURE.

“IC₅₀” refers to an amount, concentration or dosage of a particular test compound that achieves a 50% inhibition of a maximal response, such as binding, cell growth, or proliferation, measured via any of the in vitro or cell-based assays described herein.

The term “polypeptide” refers to a polymer of amino acid residues, and is not limited to a minimum length. A “protein” may comprise one or more polypeptides. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” or “protein” refers to a polypeptide or protein, respectively, which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts that produce the proteins or errors due to PCR amplification. A protein may comprise two or more polypeptides.

“PSMA” or “folate hydrolase 1” or “FOLH1,” as used herein, refers to human PSMA (huPSMA; UniProt ID: Q04609.1), unless expressly noted otherwise (i.e., murine PSMA, cynomolgus PSMA, or the like). Exemplary human PSMA amino acid sequences are shown in SEQ ID NO: 21 or SEQ ID NO: 26.

The term “antibody” herein refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term is used in the broadest sense and encompasses various antibody structures, including but not limited to monoclonal antibodies, polyclonal antibodies, multispecific antibodies (e.g., bispecific antibodies, diabodies, etc.), full length antibodies, single-chain antibodies, antibody conjugates, and antibody fragments, so long as they exhibit the desired PSMA-specific binding activity.

An “isolated” antibody is one that has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

An “antigen” refers to the target of an antibody, i.e., the molecule to which the antibody specifically binds. The term “epitope” denotes the site on an antigen, either proteinaceous or non-proteinaceous, to which an antibody binds. Epitopes on a protein can be formed both from contiguous amino acid stretches (linear epitope) or comprise non-contiguous amino acids (conformational epitope), e.g., coming in spatial proximity due to the folding of the antigen, i.e., by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents.

An “anti-PSMA antibody” or a “PSMA-antibody” or an “antibody that specifically binds to PSMA” or an “antibody that binds to PSMA” and similar phrases refer to an antibody that specifically binds to PSMA as defined herein.

The term “heavy chain” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence.

The term “complementarity determining regions” (“CDRs”) as used herein refers to each of the regions of an antibody variable region which are hypervariable in sequence and which determine antigen binding specificity. Generally, antibodies comprise six CDRs: three in the VH (CDR-H1 or heavy chain CDR1, CDR-H2, CDR-H3), and three in the VL (CDR-L1, CDR-L2, CDR-L3). Unless otherwise indicated, the CDRs are determined according to the sequence table herein.

“Framework” or “FR” refers to the residues of the variable region residues that are not part of the complementary determining regions (CDRs). The FR of a variable region generally consists of four FRs: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). See, e.g., Kindt et al. Kuby Immunology, 6^{th} ed., W.H. Freeman and Co., page 91 (2007). A variable domain may comprise heavy chain (HC) CDR1-FR2-CDR2-FR3-CDR3 with or without all or a portion of FR1 and/or FR4; and light chain (LC) CDR1-FR2-CDR2-FR3-CDR3 with or without all or a portion of FR1 and/or FR4. That is, a variable domain may lack a portion of FR1 and/or FR4 so long as it retains antigen-binding activity. A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The light chain and heavy chain “constant regions” of an antibody refer to additional sequence portions outside of the FRs and CDRs and variable regions. Certain antibody fragments may lack all or some of the constant regions. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain at Gly446 and Lys447 (EU numbering). Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine and lysine, respectively. Therefore, the C-terminal lysine, or the C-terminal glycine and lysine, of the Fc region may or may not be present. Thus, a “full-length heavy chain constant region” or a “full length antibody” for example, which is a human IgG1 antibody, includes an IgG1 with both a C-terminal glycine and lysine, without the C-terminal lysine, or without both the C-terminal glycine and lysine. Unless otherwise specified herein, numbering of amino acid residues in the Fc region or constant region is according to the EU numbering system, also called the EU index, as described in Kabat et al., Sequences of Proteins of Immunological Interest, 5^{th} Ed. Public Health Service, National Institutes of Health, Bethesda, MD, 1991.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgAQ1, and IgA2. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

An “antibody fragment” or “antigen-binding fragment” or “antigen-binding portion” refers to a fragment or portion of an antibody other than an intact antibody that binds the antigen (i.e., PSMA) to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The terms “full length antibody”, “intact antibody”, and “whole antibody” are used herein interchangeably to refer to an antibody having a structure substantially similar to a native antibody structure or, in the case of an IgG antibody, having heavy chains that contain an Fc region as defined herein above.

The term “chimeric” antibody refers to an antibody in which a portion of the heavy and/or light chain is derived from a particular source or species, while the remainder of the heavy and/or light chain is derived from a different source or species.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” as used herein refers to antibodies produced from human immunoglobulin sequences, such as antibodies produced in non-human animals that comprise human immunoglobulin genes (such as XenoMouse® and VelocImmune® mice), and antibodies selected using in vitro methods, such as phage display, wherein the antibody repertoire is based on a human immunoglobulin sequences.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method.

A “multispecific” antibody is one that binds specifically to more than one target antigen, while a “bispecific” antibody is one that binds specifically to two antigens. An “antibody conjugate” is an antibody conjugated to one or more heterologous molecule(s), including but not limited to a therapeutic agent or a label.

Antibodies may be modified as part of the production process in certain host cells or through metabolism in vivo. An antibody or antibody region amino acid sequence herein is intended to encompass not only the specific amino acid sequence, but also that sequence as post-translationally modified, for instance, including side chain modifications and cleavages. Such a post-translational modification can occur, for instance, as a result of production of the antibody in a host cell and/or as a result of post-translational modification in vivo in an animal (e.g., a human).

In some embodiments, an antibody disclosed herein comprises a post-translational modification (e.g., one or more post-translational modifications). Post-translational modifications can include, e.g., ubiquitination, phosphorylation, acetylation, hydroxylation, methylation, glycyosylation, AMPylation, prenylation, deamidation, elimylation, citrullination, and carbamoylation. In some embodiments, the antibody is not post-translationally modified.

As noted above, antibodies can undergo post-translational cleavage of one or more, particularly one or two, amino acids from the C-terminus of the heavy chain, often a Gly-Lys. This cleavage can occur, for instance, as a result of the process of production of the antibody in a host cell. An antibody produced by expression of a specific nucleic acid molecule encoding a full-length heavy chain can include the full-length heavy chain, or it can include a cleaved variant of the full-length heavy chain, such as a heavy chain lacking a C-terminal Lys or a C-terminal Gly-Lys.

Other types of post-translational modifications can occur during production of antibodies, or otherwise in vivo, such as the modification of an amino acid side chain. For instance, an N-terminal Glu or Gln residue on an antibody chain can be post-translationally modified to an N-terminal pyroglutamate (also known as pyrrolidine carboxylate; abbreviated pE).

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “signal sequence” or “leader sequence” refers to a sequence of amino acid residues located at the N terminus of a polypeptide that facilitates secretion of a polypeptide from a mammalian cell. A leader sequence may be cleaved upon export of the polypeptide from the mammalian cell, forming a mature protein. Leader sequences may be natural or synthetic, and they may be heterologous or homologous to the protein to which they are attached.

Nonlimiting exemplary leader sequences also include leader sequences from heterologous proteins. In some embodiments, an antibody lacks a leader sequence. In some embodiments, an antibody comprises at least one leader sequence, which may be selected from native antibody leader sequences and heterologous leader sequences.

The term “nucleic acid molecule” or “polynucleotide” includes any compound and/or substance that comprises a polymer of nucleotides. Each nucleotide is composed of a base, specifically a purine- or pyrimidine base (i.e. cytosine I, guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA, circular RNA) vectors, can be unmodified or modified.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-PSMA antibody” refers to one or more nucleic acid molecules encoding anti-PSMA antibody heavy and light chains (or fragments thereof), including such nucleic acid molecule(s) in a single vector or separate vectors, and such nucleic acid molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “host cell”, “host cell line”, and “host cell culture” are used interchangeably and refer to cells into which exogenous nucleic acid has been introduced, including the progeny of such cells.

In this disclosure, “binds” or “binding” or “specific binding” and similar terms, when referring to a protein and its ligand or an antibody and its antigen target for example, or some other binding pair, means that the binding affinity between the members of the binding pair is sufficiently strong that the interaction cannot be due to random molecular associations (i.e. “nonspecific binding”). Such binding typically requires a dissociation constant (K_{D}) of 1 μM or less, and may often involve a K_{D} of 100 nM or less.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). Affinity can generally be represented by the dissociation constant (K_{D}). Affinity of an antibody for an antigen can be measured by common methods known in the art, such as biolayer interferometry or surface plasmon resonance (SPR), for instance. Unless otherwise indicated, K_{D} values provided herein were determined using biolayer interferometry.

The terms “reduce” or “inhibit” more generally refer to a decrease or cessation of any event (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

“Treatment” or “treating” as used herein, covers any administration or application of a therapeutic for disease in a human, and includes inhibiting the disease or progression of the disease or one or more disease symptoms, inhibiting or slowing the disease or its progression or one or more of its symptoms, arresting its development, partially or fully relieving the disease or one or more of its symptoms, or preventing a recurrence of one or more symptoms of the disease.

The terms “subject” and “patient” are used interchangeably herein to refer to a human unless expressly indicated otherwise (i.e., a murine subject or the like).

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells or leukemic cancer cells. In some embodiments, a cancer is a solid cancer.

Nonlimiting exemplary cancers include but are not limited to prostate cancer, castration resistant prostate cancer (CRPC), and metastatic castration resistant prostate cancer (mCRPC).

As used herein, the term “tumor,” refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth.

“Anti-cancer agents” refers to anti-metabolites (e.g., 5-fluoro-uracil, methotrexate, fludarabine), antimicrotubule agents (e.g., vinca alkaloids such as vincristine, vinblastine; taxanes such as paclitaxel, docetaxel), alkylating agents (e.g., cyclophosphamide, melphalan, carmustine, nitrosoureas such as bischloroethylnitrosurea and hydroxyurea), platinum agents (e.g. cisplatin, carboplatin, oxaliplatin, JM-216 or satraplatin, CI-973), anthracyclines (e.g., doxorubicin, daunorubicin), antitumor antibiotics (e.g., mitomycin, idarubicin, adriamycin, daunomycin), topoisomerase inhibitors (e.g., etoposide, camptothecins), anti-angiogenesis agents (e.g. Sutent® and Bevacizumab) or any other cytotoxic agents, (estramustine phosphate, prednimustine), hormones or hormone agonists, antagonists, partial agonists or partial antagonists, kinase inhibitors, and radiation treatment.

The term “effective amount” or “therapeutically effective amount” refers to an amount of a drug effective for treatment of a disease or disorder in a subject, such as to partially or fully relieve one or more symptoms. In some embodiments, an effective amount refers to an amount effective, at dosages and for periods of time necessary, to achieve the desired therapeutic or prophylactic result.

A “biological sample” as used herein refers to a sample taken from a subject or from an animal. Examples of biological samples include tissue samples and liquid biological samples, such as whole blood, serum, plasma, blood supernatant, or synovial fluid. A biological sample may be taken directly from a subject or may be first chemically or physically modified in some fashion prior to use, for example, in order to assist in analysis of the sample.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

I. Exemplary Anti-PSMA Antibodies Novel antibodies directed against PSMA, such as human PSMA, are provided. Anti-PSMA antibodies include, but are not limited to, humanized antibodies, chimeric antibodies, mouse antibodies, human antibodies, and antibodies comprising the heavy chain and/or light chain CDRs discussed herein. In some embodiments, an isolated antibody that binds to PSMA is provided. In some embodiments, a monoclonal antibody that binds to PSMA is provided. In some embodiments, the antibody binds to human PSMA. In some embodiments, the antibody binds to human PSMA comprising the amino acid sequence of SEQ ID NO: 21 or SEQ ID NO: 26. In some embodiments, the antibody binds to cynomolgus monkey PSMA comprising the amino acid sequence of SEQ ID NO: 22.

In some embodiments, an anti-PSMA antibody comprises a heavy chain variable region and a light chain variable region. In some embodiments, an anti-PSMA antibody comprises at least one heavy chain comprising a heavy chain variable region and at least a portion of a heavy chain constant region, and at least one light chain comprising a light chain variable region and at least a portion of a light chain constant region. In some embodiments, an anti-PSMA antibody comprises two heavy chains, wherein each heavy chain comprises a heavy chain variable region and at least a portion of a heavy chain constant region, and two light chains, wherein each light chain comprises a light chain variable region and at least a portion of a light chain constant region. As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some embodiments, the heavy chain is the region of the anti-PSMA antibody that comprises the three heavy chain CDRs. In some embodiments, the light chain is the region of the anti-PSMA antibody that comprises the three light chain CDRs.

In some embodiments, an anti-PSMA antibody comprises at least one, two, three, four, five, or six CDRs selected from a heavy chain complementarity determining region 1 (HCDR1) comprising an amino acid sequence selected from SEQ ID NOs: 3-5; a heavy chain complementarity determining region 2 (HCDR2) comprising an amino acid sequence selected from SEQ ID NOs: 6-9; a heavy chain complementarity determining region 3 (HCDR3) comprising the amino acid sequence of SEQ ID NO: 10 or 11; a light chain complementarity determining region 1 (LCDR1) comprising the amino acid sequence of SEQ ID NO: 12 or 13; a light chain complementarity determining region 2 (LCDR2) comprising the amino acid sequence of SEQ ID NO: 14 or 15; and a light chain complementarity determining region 3 (LCDR3) comprising the amino acid sequence of SEQ ID NO: 16.

In some embodiments, the anti-PSMA antibody comprises six CDRs including a heavy chain complementarity determining region 1 (HCDR1) comprising an amino acid sequence selected from SEQ ID NOs: 3-5; a heavy chain complementarity determining region 2 (HCDR2) comprising an amino acid sequence selected from SEQ ID NOs: 6-9; a heavy chain complementarity determining region 3 (HCDR3) comprising the amino acid sequence of SEQ ID NO: 10 or 11; a light chain complementarity determining region 1 (LCDR1) comprising the amino acid sequence of SEQ ID NO: 12 or 13; a light chain complementarity determining region 2 (LCDR2) comprising the amino acid sequence of SEQ ID NO: 14 or 15; and a light chain complementarity determining region 3 (LCDR3) comprising the amino acid sequence of SEQ ID NO: 16.

In some embodiments, the anti-PSMA antibody comprises six CDRs including HCDR1 comprising the amino acid sequence of SEQ ID NO: 3, HCDR2 comprising the amino acid sequence of SEQ ID NO: 6, HCDR3 comprising the amino acid sequence of SEQ ID NO: 10, LCDR1 comprising the amino acid sequence of SEQ ID NO: 12, LCDR2 comprising the amino acid sequence of SEQ ID NO: 14, LCDR3 comprising the amino acid sequence of SEQ ID NO: 16.

In some embodiments, the anti-PSMA antibody comprises six CDRs including HCDR1 comprising the amino acid sequence of SEQ ID NO: 4, HCDR2 comprising the amino acid sequence of SEQ ID NO: 7, HCDR3 comprising the amino acid sequence of SEQ ID NO: 10, LCDR1 comprising the amino acid sequence of SEQ ID NO: 12, LCDR2 comprising the amino acid sequence of SEQ ID NO: 14, LCDR3 comprising the amino acid sequence of SEQ ID NO: 16.

In some embodiments, the anti-PSMA antibody comprises six CDRs including HCDR1 comprising the amino acid sequence of SEQ ID NO: 4, HCDR2 comprising the amino acid sequence of SEQ ID NO: 8, HCDR3 comprising the amino acid sequence of SEQ ID NO: 11, LCDR1 comprising the amino acid sequence of SEQ ID NO: 13, LCDR2 comprising the amino acid sequence of SEQ ID NO: 15, LCDR3 comprising the amino acid sequence of SEQ ID NO: 16.

In some embodiments, the anti-PSMA antibody comprises six CDRs including HCDR1 comprising the amino acid sequence of SEQ ID NO: 5, HCDR2 comprising the amino acid sequence of SEQ ID NO: 9, HCDR3 comprising the amino acid sequence of SEQ ID NO: 10, LCDR1 comprising the amino acid sequence of SEQ ID NO: 12, LCDR2 comprising the amino acid sequence of SEQ ID NO: 14, LCDR3 comprising the amino acid sequence of SEQ ID NO: 16.

In some embodiments, any of the six CDRs provided herein can be combined as subparts with any of the other CDRs provided herein, for a total of six CDRs in a construct. Thus, in some embodiments, two CDRs from a first antibody (for example, HCDR1 and HCDR2) can be combined with four CDRs from a second antibody (HCDR3, LCDR1, LCDR2, and LCDR3). In some embodiments, two or fewer residues in one or more of the CDRs can be replaced to obtain a variant thereof. In some embodiments, two or fewer residues can be replaced in 1, 2, 3, 4, 5, or 6 of the CDRs.

In some embodiments, the anti-PSMA antibody comprises (I) a VH domain comprising at least one, at least two, or all three VH CDR sequences selected from (i) an HCDR1 comprising an amino acid sequence selected from SEQ ID NOs: 3-5; (ii) an HCDR2 comprising an amino acid sequence selected from SEQ ID NOs: 6-9; (iii) an HCDR3 comprising the amino acid sequence of SEQ ID NO: 10 or 11; and (II) a VL domain comprising at least one, at least two, or all three VL CDR sequences selected from (iv) an LCDR1 comprising the amino acid sequence of SEQ ID NO: 12 or 13; (v) an LCDR2 comprising the amino acid sequence of SEQ ID NO: 14; and (vi) an LCDR3 comprising the amino acid sequence of SEQ ID NO: 16; wherein the VH domain comprises a sequence having at least 90%, 91%, 92%, 93%, 94%, 95%, 96%, 97%, 98%, 99%, or 100% sequence identity to the amino acid sequence of SEQ ID NO: 1, and the VL domain comprises a sequence having at least 90%, 91%, 92%, 93%, 94%, 95%, 96%, 97%, 98%, 99%, or 100% sequence identity to the amino acid sequence of SEQ ID NO: 2.

In some embodiments, an anti-PSMA antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 1. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, but an anti-PSMA antibody comprising that sequence retains the ability to bind PSMA. In some embodiments, a total of 1 to 10 amino acids have been substituted, inserted and/or deleted in a SEQ ID NO: 1. In some embodiments, substitutions, insertions, or deletions occur in regions outside the CDRs (that is, in the FRs). Optionally, the anti-PSMA antibody comprises the VH sequence of SEQ ID NO: 1, including post-translational modifications of that sequence.

In some embodiments, an anti-PSMA antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 2.

In some embodiments, a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-PSMA antibody comprising that sequence retains the ability to bind to PSMA. In some embodiments, a total of 1 to 10 amino acids have been substituted, inserted and/or deleted in SEQ ID NO: 2. In some embodiments, the substitutions, insertions, or deletions occur in regions outside the CDRs (that is, in the FRs). Optionally, the anti-PSMA antibody comprises the VL sequence of SEQ ID NO: 2, including post-translational modifications of that sequence.

In some embodiments, an anti-PSMA antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 1 and a light chain variable domain (VL) having at least 90%, 91%, 92%, 93%, 94%, 95%, 96%, 97%, 98%, 99%, or 100% sequence identity to the amino acid sequence of SEQ ID NO: 2. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, and a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-PSMA antibody comprising that sequence retains the ability to bind to PSMA. In some embodiments, a total of 1 to 10 amino acids have been substituted, inserted and/or deleted in SEQ ID NO: 1. In some embodiments, a total of 1 to 10 amino acids have been substituted, inserted and/or deleted in SEQ ID NO: 2. In some embodiments, substitutions, insertions, or deletions occur in regions outside the CDRs (that is, in the FRs). Optionally, the anti-PSMA antibody comprises the VH sequence of SEQ ID NO: 1 and the VL sequence of SEQ ID NO: 2, including post-translational modifications of one or both sequences.

In some embodiments, an anti-PSMA antibody comprises a VH as in any of the embodiments provided herein, and a VL as in any of the embodiments provided herein. In some embodiments, the antibody comprises a VH comprising the amino acid sequence of SEQ ID NO: 1 and a VL comprising the amino acid sequence of SEQ ID NO: 2, including post-translational modifications of those sequences.

In various embodiments, an anti-PSMA antibody provided herein may comprise pyroglutamic acid (pyroGlu or pE) in place of glutamic acid (Glu or E) at the N-terminus. In some embodiments, pyroGlu is formed by cyclization of an N-terminal Glu during expression and/or purification of the antibody. Thus, a Glu (E) at the N-terminus of an antibody chain sequence provided herein may be replaced with a pyroGlu, and antibodies comprising pyroGlu in place of Glu at the N-terminus the sequences provided herein are contemplated. Thus, in some embodiments, an anti-PSMA antibody is provided, wherein the antibody comprises a heavy chain variable domain comprising the sequence of any one of SEQ ID NO: 1, wherein the N-terminal Glu (E) is replaced with a pyroGlu (pE). Similarly, an anti-PSMA antibody is provided, wherein the antibody comprises a heavy chain comprising the sequence of any one of SEQ ID NO: 17, wherein the N-terminal Glu (E) is replaced with a pyroGlu (pE).

In some embodiments, the anti-PSMA antibody comprises six CDRs including HCDR1 comprising the amino acid sequence of SEQ ID NO: 29, 30, 31, or 32, HCDR2 comprising the amino acid sequence of SEQ ID NO: 33, 34, 35, or 36, HCDR3 comprising the amino acid sequence of SEQ ID NO: 37 or 38, LCDR1 comprising the amino acid sequence of SEQ ID NO: 39 or 40, LCDR2 comprising the amino acid sequence of SEQ ID NO: 41 or 42, and LCDR3 comprising the amino acid sequence of SEQ ID NO: 43.

In some embodiments, the anti-PSMA antibody comprises six CDRs including HCDR1 comprising the amino acid sequence of SEQ ID NO: 29, HCDR2 comprising the amino acid sequence of SEQ ID NO: 33, HCDR3 comprising the amino acid sequence of SEQ ID NO: 37, LCDR1 comprising the amino acid sequence of SEQ ID NO: 39, LCDR2 comprising the amino acid sequence of SEQ ID NO:41, and LCDR3 comprising the amino acid sequence of SEQ ID NO:43.

In some embodiments, the anti-PSMA antibody comprises six CDRs including HCDR1 comprising the amino acid sequence of SEQ ID NO: 30, HCDR2 comprising the amino acid sequence of SEQ ID NO: 34, HCDR3 comprising the amino acid sequence of SEQ ID NO: 37, LCDR1 comprising the amino acid sequence of SEQ ID NO:39, LCDR2 comprising the amino acid sequence of SEQ ID NO:41, and LCDR3 comprising the amino acid sequence of SEQ ID NO:43.

In some embodiments, the anti-PSMA antibody comprises six CDRs including HCDR1 comprising the amino acid sequence of SEQ ID NO: 31, HCDR2 comprising the amino acid sequence of SEQ ID NO: 35, HCDR3 comprising the amino acid sequence of SEQ ID NO: 38, LCDR1 comprising the amino acid sequence of SEQ ID NO:40, LCDR2 comprising the amino acid sequence of SEQ ID NO:42, and LCDR3 comprising the amino acid sequence of SEQ ID NO:43.

In some embodiments, the anti-PSMA antibody comprises six CDRs including HCDR1 comprising the amino acid sequence of SEQ ID NO: 32, HCDR2 comprising the amino acid sequence of SEQ ID NO: 36, HCDR3 comprising the amino acid sequence of SEQ ID NO: 37, LCDR1 comprising the amino acid sequence of SEQ ID NO:39, LCDR2 comprising the amino acid sequence of SEQ ID NO:41, and LCDR3 comprising the amino acid sequence of SEQ ID NO:43.

In some embodiments, the anti-PSMA antibody comprises six CDRs including HCDR1 comprising the amino acid sequence of SEQ ID NO: 29, 30, 31, or 32, HCDR2 comprising the amino acid sequence of SEQ ID NO: 33, 34, 35, or 36, HCDR3 comprising the amino acid sequence of SEQ ID NO: 37 or 38, LCDR1 comprising the amino acid sequence of SEQ ID NO: 39 or 40, LCDR2 comprising the amino acid sequence of SEQ ID NO: 41 or 42, and LCDR3 comprising the amino acid sequence of SEQ ID NO: 43, wherein the VH domain comprises a sequence having at least 90%, 91%, 92%, 93%, 94%, 95%, 96%, 97%, 98%, 99%, or 100% sequence identity to the amino acid sequence of SEQ ID NO: 27 (PSMA-Ab3), and the VL domain comprises a sequence having at least 90%, 91%, 92%, 93%, 94%, 95%, 96%, 97%, 98%, 99%, or 100%, identity to the amino acid sequence of SEQ ID NO: 28 (PSMA-Ab3).

In some embodiments, an anti-PSMA antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 27 (PSMA-Ab3). In some embodiments, an anti-PSMA antibody comprises a heavy chain variable domain (VH) sequence comprising an amino acid sequence set forth in SEQ ID NO: 27. In some embodiments, an anti-PSMA antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 28 (PSMA-Ab3). In some embodiments, an anti-PSMA antibody comprises a light chain variable domain (VL) sequence comprising the amino acid sequence set forth in SEQ ID NO: 28. In some embodiments, an anti-PSMA antibody comprises a heavy chain variable domain (VH) sequence comprising an amino acid sequence set forth in SEQ ID NO: 27 and a light chain variable domain (VL) sequence comprising the amino acid sequence set forth in SEQ ID NO: 28, including post-translational modifications of one or both sequences.

In some embodiments, the anti-PSMA antibody comprises six CDRs including HCDR1 comprising the amino acid sequence of SEQ ID NO: 29, 30, 31, or 32, HCDR2 comprising the amino acid sequence of SEQ ID NO: 33, 34, 35, or 36, HCDR3 comprising the amino acid sequence of SEQ ID NO: 37 or 38, LCDR1 comprising the amino acid sequence of SEQ ID NO: 39 or 40, LCDR2 comprising the amino acid sequence of SEQ ID NO: 41 or 42, and LCDR3 comprising the amino acid sequence of SEQ ID NO: 43; wherein the heavy chain (HC) sequence comprises at least 90%, 91%, 92%, 93%, 94%, 95%, 96%, 97%, 98%, 99%, or 100% sequence identity to the amino acid sequence of SEQ ID NO: 44 (PSMA-Ab3) and the light chain (LC) sequence comprises at least 90%, 91%, 92%, 93%, 94%, 95%, 96%, 97%, 98%, 99%, or 100% sequence identity to the amino acid sequence of SEQ ID NO: 45 (PSMA-Ab3).

In some embodiments, an anti-PSMA antibody comprises a heavy chain (HC) sequence having at least 90%, 91%, 92%, 93%, 94%, 95%, 96%, 97%, 98%, 99%, or 100% sequence identity to the amino acid sequence of SEQ ID NO: 44 (PSMA-Ab3). In some embodiments, the antibody comprises a heavy chain (HC) comprising the amino acid sequence of SEQ ID NO: 44 (PSMA-Ab3 heavy chain). In some embodiments, an anti-PSMA antibody comprises a light chain (LC) sequence having at least 90%, 91%, 92%, 93%, 94%, 95%, 96%, 97%, 98%, 99%, or 100% sequence identity to the amino acid sequence of SEQ ID NO: 45 (PSMA-Ab3). In some embodiments, the antibody comprises a light chain (LC) comprising the amino acid sequence selected of SEQ ID NO: 45 (PSMA-Ab3 light chain). In some embodiments, the antibody comprises a heavy chain (HC) comprising the amino acid sequence of SEQ ID NO: 44 (PSMA-Ab3 heavy chain) and a light chain (LC) comprising the amino acid sequence selected of SEQ ID NO: 45 (PSMA-Ab3 light chain).

In some embodiments, the anti-PSMA antibody comprises six CDRs including HCDR1 comprising the amino acid sequence of SEQ ID NO:48, HCDR2 comprising the amino acid sequence of SEQ ID NO: 49, HCDR3 comprising the amino acid sequence of SEQ ID NO:50, LCDR1 comprising the amino acid sequence of SEQ ID NO:51, LCDR2 comprising the amino acid sequence of SEQ ID NO:52, and LCDR3 comprising the amino acid sequence of SEQ ID NO:53.

In some embodiments, the anti-PSMA antibody comprises six CDRs including HCDR1 comprising the amino acid sequence of SEQ ID NO:48, HCDR2 comprising the amino acid sequence of SEQ ID NO: 49, HCDR3 comprising the amino acid sequence of SEQ ID NO:50, LCDR1 comprising the amino acid sequence of SEQ ID NO:51, LCDR2 comprising the amino acid sequence of SEQ ID NO:52, and LCDR3 comprising the amino acid sequence of SEQ ID NO:53, wherein the VH domain comprises a sequence having at least 90%, 91%, 92%, 93%, 94%, 95%, 96%, 97%, 98%, 99%, or 100% sequence identity to the amino acid sequence of SEQ ID NO: 46 (PSMA-Ab4), and the VL domain comprises a sequence having at least 90%, 91%, 92%, 93%, 94%, 95%, 96%, 97%, 98%, 99%, or 100%, identity to the amino acid sequence of SEQ ID NO: 47 (PSMA-Ab4).

In some embodiments, an anti-PSMA antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 46 (PSMA-Ab4). In some embodiments, an anti-PSMA antibody comprises a heavy chain variable domain (VH) sequence comprising an amino acid sequence set forth in SEQ ID NO: 46. In some embodiments, an anti-PSMA antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 47 (PSMA-Ab4). In some embodiments, an anti-PSMA antibody comprises a light chain variable domain (VL) sequence comprising the amino acid sequence set forth in SEQ ID NO: 47. In some embodiments, an anti-PSMA antibody comprises a heavy chain variable domain (VH) sequence comprising an amino acid sequence set forth in SEQ ID NO: 46 and a light chain variable domain (VL) sequence comprising the amino acid sequence set forth in SEQ ID NO: 47,including post-translational modifications of one or both sequences.

In some embodiments, the anti-PSMA antibody comprises six CDRs including HCDR1 comprising the amino acid sequence of SEQ ID NO:48, HCDR2 comprising the amino acid sequence of SEQ ID NO: 49, HCDR3 comprising the amino acid sequence of SEQ ID NO:50, LCDR1 comprising the amino acid sequence of SEQ ID NO:51, LCDR2 comprising the amino acid sequence of SEQ ID NO:52, and LCDR3 comprising the amino acid sequence of SEQ ID NO:53, wherein the heavy chain (HC) sequence comprises at least 90%, 91%, 92%, 93%, 94%, 95%, 96%, 97%, 98%, 99%, or 100% sequence identity to the amino acid sequence of SEQ ID NO: 54 (PSMA-Ab4 heavy chain), and the light chain (LC) sequence comprises at least 90%, 91%, 92%, 93%, 94%, 95%, 96%, 97%, 98%, 99%, or 100% sequence identity to the amino acid sequence of SEQ ID NO: 55 (PSMA-Ab4 light chain).

In some embodiments, an anti-PSMA antibody comprises a heavy chain (HC) sequence having at least 90%, 91%, 92%, 93%, 94%, 95%, 96%, 97%, 98%, 99%, or 100% sequence identity to the amino acid sequence of SEQ ID NO: 54 (PSMA-Ab4 heavy chain). In some embodiments, the antibody comprises a heavy chain (HC) comprising the amino acid sequence of SEQ ID NO: 54 (PSMA-Ab4 heavy chain). In some embodiments, an anti-PSMA antibody comprises a light chain (LC) sequence having at least 90%, 91%, 92%, 93%, 94%, 95%, 96%, 97%, 98%, 99%, or 100% sequence identity to the amino acid sequence of SEQ ID NO: 55 (PSMA-Ab4 light chain). In some embodiments, the antibody comprises a light chain (LC) comprising the amino acid sequence selected of SEQ ID NO: 55 (PSMA-Ab4 light chain). In some embodiments, the antibody comprises a heavy chain (HC) comprising the amino acid sequence of SEQ ID NO: 54 (PSMA-Ab4 heavy chain) and a light chain (LC) sequence comprising the amino acid sequence of SEQ ID NO: 55 (PSMA-Ab4 light chain).

In some embodiments, the anti-PSMA antibody comprises six CDRs including HCDR1 comprising the amino acid sequence of SEQ ID NO:58, HCDR2 comprising the amino acid sequence of SEQ ID NO: 59, HCDR3 comprising the amino acid sequence of SEQ ID NO:60, LCDR1 comprising the amino acid sequence of SEQ ID NO:61, LCDR2 comprising the amino acid sequence of SEQ ID NO:62, and LCDR3 comprising the amino acid sequence of SEQ ID NO:63.

In some embodiments, the anti-PSMA antibody comprises six CDRs including HCDR1 comprising the amino acid sequence of SEQ ID NO:58, HCDR2 comprising the amino acid sequence of SEQ ID NO: 59, HCDR3 comprising the amino acid sequence of SEQ ID NO:60, LCDR1 comprising the amino acid sequence of SEQ ID NO:61, LCDR2 comprising the amino acid sequence of SEQ ID NO:62, and LCDR3 comprising the amino acid sequence of SEQ ID NO:63, wherein the VH domain comprises a sequence having at least 90%, 91%, 92%, 93%, 94%, 95%, 96%, 97%, 98%, 99%, or 100% sequence identity to the amino acid sequence of SEQ ID NO:56 (PSMA-Ab5) and the VL domain comprises a sequence having at least 90%, 91%, 92%, 93%, 94%, 95%, 96%, 97%, 98%, 99%, or 100%, identity to the amino acid sequence of SEQ ID NO: 57 (PSMA-Ab5).

In some embodiments, an anti-PSMA antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 56 (PSMA-Ab5). In some embodiments, an anti-PSMA antibody comprises a heavy chain variable domain (VH) sequence comprising an amino acid sequence set forth in SEQ ID NO: 56. In some embodiments, an anti-PSMA antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 57 (PSMA-Ab5). In some embodiments, an anti-PSMA antibody comprises a light chain variable domain (VL) sequence comprising the amino acid sequence set forth in SEQ ID NO: 57. In some embodiments, an anti-PSMA antibody comprises a heavy chain variable domain (VH) sequence comprising an amino acid sequence set forth in SEQ ID NO: 56 and a light chain variable domain (VL) sequence comprising the amino acid sequence set forth in SEQ ID NO: 57, including post-translational modifications of one or both sequences.

In some embodiments, the anti-PSMA antibody comprises six CDRs including HCDR1 comprising the amino acid sequence of SEQ ID NO:58, HCDR2 comprising the amino acid sequence of SEQ ID NO: 59, HCDR3 comprising the amino acid sequence of SEQ ID NO:60, LCDR1 comprising the amino acid sequence of SEQ ID NO:61, LCDR2 comprising the amino acid sequence of SEQ ID NO:62, and LCDR3 comprising the amino acid sequence of SEQ ID NO:63, wherein the heavy chain (HC) sequence comprises at least 90%, 91%, 92%, 93%, 94%, 95%, 96%, 97%, 98%, 99%, or 100% sequence identity to the amino acid sequence of SEQ ID NO: 64 (PSMA-Ab5 heavy chain) and the light chain (LC) sequence comprises at least 90%, 91%, 92%, 93%, 94%, 95%, 96%, 97%, 98%, 99%, or 100% sequence identity to the amino acid sequence of SEQ ID NO: 65 (PSMA-Ab5 light chain).

In some embodiments, an anti-PSMA antibody comprises a heavy chain (HC) sequence having at least 90%, 91%, 92%, 93%, 94%, 95%, 96%, 97%, 98%, 99%, or 100% sequence identity to the amino acid sequence of SEQ ID NO: 64 (PSMA-Ab5 heavy chain). In some embodiments, the antibody comprises a heavy chain (HC) comprising the amino acid sequence of SEQ ID NO: 64 (PSMA-Ab5 heavy chain). In some embodiments, an anti-PSMA antibody comprises a light chain (LC) sequence having at least 90%, 91%, 92%, 93%, 94%, 95%, 96%, 97%, 98%, 99%, or 100% sequence identity to the amino acid sequence of SEQ ID NO: 65 (PSMA-Ab5 light chain). In some embodiments, the antibody comprises light chain (LC) sequence comprising the amino acid sequence of SEQ ID NO: 65 (PSMA-Ab5 light chain). In some embodiments, the antibody comprises a heavy chain (HC) comprising the amino acid sequence of SEQ ID NO: 64 and light chain (LC) comprising the amino acid sequence selected of SEQ ID NO: 65.

In various embodiments, the antibody is a monoclonal antibody.

In various embodiments, an anti-PSMA antibody binds to human PSMA with an affinity (K_{D}) of less than about 10 nM or less than about 5 nM, from about 0.1 nM to about 10 nM, from about 0.1 nM to about 5 nM, from about 0.5 nM to about 10 nM, from about 0.5 nM to about 5 nM, from about 1 nM to about 10 nM, or from about 1 nM to about 5 nM. In some embodiments, the antibody binds to cynomolgus monkey PSMA with a K_{D} less than about 10 nM or less than about 7 nM, from about 0.1 nM to about 10 nM, from about 0.1 nM to about 7 nM, from about 0.5 nM to about 10 nM, from about 0.5 nM to about 7 nM, from about 1 nM to about 10 nM, or from about 1 nM to about 7 nM. In some embodiments, the antibody binds to human PSMA with a K_{D} of less than about 5 nM and binds to cynomolgus monkey PSMA with an affinity (K_{D}) of less than about 7 nM. In some embodiments, the anti-PSMA antibody binds to canine (dog) PSMA with an affinity (K_{D}) of less than 200 nM, less than 150 nM, less than 125 nM, from about 50 nM to about 200 nM, from about 50 nM to 150 nM, or from about 50 nM to about 125 nM. In some embodiments, affinity is determined using surface plasmon resonance (SPR). In some embodiments, the anti-PSMA antibody does not bind to rat PSMA and/or mouse PSMA. In some embodiments, the anti-PSMA antibody does not bind to either rat PSMA or mouse PSMA.

In some embodiments, the antibody binds human PSMA. In some embodiments, the human PSMA comprises the amino acid sequence of SEQ ID NO: 21 or SEQ ID NO: 26. In some embodiments, the antibody binds cynomolgus monkey PSMA. In some embodiments, the cynomolgus monkey PSMA comprises the amino acid sequence of SEQ ID NO: 22.

### II. Exemplary Antibody Variants, Fragments, and Constant Regions

In many embodiments, an antibody specifically binding to PSMA may further incorporate any of the features, singly or in combination, as described in the sections that follow.

### A. Antibody Fragments

In certain embodiments, an antibody provided herein is an antibody fragment. In some embodiments, the anti-PSMA antibody is an antibody fragment selected from an Fv, single-chain Fv (scFv), Fab, Fab′, or (Fab′)₂. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,87,458. For discussion of Fab and F(a″)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### B. Bispecific or Multispecific Antibodies

In certain embodiments, an antibody provided herein is a multispecific antibody, for example, a bispecific antibody. Multispecific antibodies are monoclonal antibodies that have binding specificities for at least two different sites. In certain embodiments, one of the binding specificities is PSMA and the other is for any other antigen. In certain embodiments, bispecific antibodies may bind to two different epitopes of PSMA. Bispecific antibodies may also be used to localize drugs such as cytotoxic agents or to localize detection labels to cells that express PSMA. some embodiments, the multispecific antibody (e.g., bispecific antibody) comprises a first variable domain comprising the CDRs or variable regions as described herein. Bispecific antibodies can be prepared as full length antibodies or antibody fragments.

In some embodiments, an antibody provided herein is a bispecific antibody comprising a first antigen-binding domain that binds PSMA and a second antigen-binding domain that binds a second antigen. In various embodiments, the first antigen-binding domain is an antigen-binding domain that binds PSMA described herein.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 18(5):1547-1553 (1992)); using “diabod” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576).

### C. Human Antibodies

In certain embodiments, an antibody is a human antibody. Human antibodies can be made by any suitable method. Nonlimiting exemplary methods include making human antibodies in transgenic mice that comprise human immunoglobulin loci. See, e.g., Jakobovits et al., Proc. Natl. Acad. Sci. USA 90: 2551-55 (1993); Jakobovits et al., Nature 362: 255-8 (1993); Lonberg et al., Nature 368: 856-9 (1994); and U.S. Pat. Nos. 5,545,807; 6,713,610; 6,673,986; 6,162,963; 5,545,807; 6,300,129; 6,255,458; 5,877,397; 5,874,299; and 5,545,806.

Nonlimiting exemplary methods also include selecting human antibodies from phage display libraries. See, e.g., Hoogenboom et al., J. Mol. Biol. 227: 381-8 (1992); Marks et al., J. Mol. Biol. 222: 581-97 (1991); and PCT Publication No. WO 99/10494.

### D. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. In some embodiments, an antibody provided herein is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences.

A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carte r et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In some embodiments, the humanized antibodies may comprise a human IgG1, IgG2, IgG3, or IgG4 heavy chain constant region.

### E. Glycosylation and Pegylation Variants

In certain embodiments, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in further detail in U.S. Pat. Nos. 5,714,350 and 6,350,861 by Co et al.

Glycosylation of the constant region on N297 can be prevented by mutating the N297 residue to another residue, e.g., N297A, and/or by mutating an adjacent amino acid, e.g., 298 to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Led 3 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases {e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17: 176-180).

Another modification of the antibodies described herein is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. In some embodiments, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies described herein. See for example, EP 0 154 316 by Nishimura et al. and EP 0 401 384 by Ishikawa et al.

### F. Constant Regions

In some embodiments, the antibody is a full-length antibody. In some embodiments, an antibody described herein comprises one or more human constant regions. In some embodiments, the human heavy chain constant region is of an isotype selected from IgA, IgG, and IgD. In some embodiments, the human light chain constant region is of an isotype selected from κ and λ. In some embodiments, the antibody is an IgG antibody. In some embodiments, the antibody is an IgG antibody lacking a C-terminal lysine in the heavy chain constant region. In some embodiments, an antibody described herein comprises a human IgG constant region, such as an IgG1, IgG2, IgG3, or IgG4. In some embodiments, the antibody is an IgG1 antibody, an IgG2 antibody, an IgG3 antibody, or an IgG4 antibody. In some embodiments, an antibody described herein comprises a human IgG4 heavy chain constant region. In some such embodiments, an antibody described herein comprises an S241P mutation in the human IgG4 constant region. In some embodiments, an antibody described herein comprises a human IgG4 constant region and a human κ light chain.

In some embodiments, a heavy chain constant region of an antibody provided herein comprises the amino acid sequence of SEQ ID NO: 19. In some embodiments, a light chain constant region of an antibody provided herein comprises the amino acid sequence of SEQ ID NO: 20.

In some embodiments, a human IgG heavy chain Fc region extends from Cys226, or from Pro230, to the carboxyl-terminus of the heavy chain at Gly446 and Lys447 (EU numbering). Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain and/or a cleaved variant of the full-length heavy chain. In some embodiments, the C-terminal lysine, or the C-terminal glycine and lysine, of the Fc region may or may not be present. Thus, a “full-length heavy chain constant region” or a “full length antibody” for example, which is a human IgG1 antibody, includes an IgG1 with both a C-terminal glycine and lysine, without the C-terminal lysine, or without both the C-terminal glycine and lysine.

The choice of heavy chain constant region can determine whether or not an antibody will have effector function in vivo. Such effector function, in some embodiments, includes antibody-dependent cell-mediated cytotoxicity (ADCC) and/or complement-dependent cytotoxicity (CDC), and can result in killing of the cell to which the antibody is bound. In some methods of treatment, including methods of treating some cancers, cell killing may be desirable, for example, when the antibody binds to a cell that supports the maintenance or growth of the tumor. Exemplary cells that may support the maintenance or growth of a tumor include, but are not limited to, tumor cells themselves, cells that aid in the recruitment of vasculature to the tumor, and cells that provide ligands, growth factors, or counter-receptors that support or promote tumor growth or tumor survival. In some embodiments, when effector function is desirable, an antibody comprising a human IgG1 heavy chain or a human IgG3 heavy chain is selected.

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibodies with certain improved properties. For example, in some embodiments an antibody may be afucosylated, for example, by mutating residues such as Asn297 that are normally glycosylated with fucose-containing glycosylations, or through other means. In some embodiments, antibodies herein may comprise an afucosylated human IgG1 constant region.

Antibodies are further provided with bisected oligosaccharides, e.g., in which a biantennary oligosaccharide attached to the Fc region of the antibody is bisected by GlcNAc. Such antibodies may have reduced fucosylation and/or improved ADCC function. Examples of such antibodies are described, e.g., in WO 2003/011878 (Jean-Mairet et al.); U.S. Pat. No. 6,602,684 (Umana et al.); and US 2005/0123546 (Umana et al.). Antibodies with at least one galactose residue in the oligosaccharide attached to the Fc region are also provided. Such antibodies may have improved CDC function. Such antibodies are described, e.g., in WO 1997/30087 (Patel et al.); WO 1998/58964 (Raju, S.); and WO 1999/22764 (Raju, S.).

Antibodies are also provided with amino-terminal leader extensions. For example, one or more amino acid residues of the amino-terminal leader sequence are present at the amino-terminus of any one or more heavy or light chains of an antibody. An exemplary amino-terminal leader extension comprises or consists of three amino acid residues, VHS, present on one or both light chains of an antibody.

The in vivo or serum half-life of human FcRn high affinity binding polypeptides can be assayed, e.g., in transgenic mice, in humans, or in non-human primates to which the polypeptides with a variant Fc region are administered. See also, e.g., Petkova et al. International Immunology 18(12):1759-1769 (2006).

In some embodiments of the invention, an afucosylated antibody mediates ADCC in the presence of human effector cells more effectively than a parent antibody that comprises fucose, Generally, ADCC activity may be determined using the in vitro ADCC assay as herein disclosed, but other assays or methods for determining ADCC activity, e.g. in an animal model etc., are contemplated.

In certain embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320, 322, 330, and/or 331 (EU numbering) can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In some examples, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In some examples, one or more amino acid residues within amino acid positions 231 and 239 are altered to thereby alter the ability of the antibody to fix complement. This approach is described further in PCT Publication WO 94/29351 by Bodmer et al. In some examples, the Fc region can be modified to decrease antibody dependent cellular cytotoxicity (ADCC) and/or to decrease the affinity for an Fc7 receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439 (EU numbering). Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F7324T (EU numbering). Other Fc modifications that can be made to Fcs are those for reducing or ablating binding to FcγR and/or complement proteins, thereby reducing or ablating Fc-mediated effector functions such as ADCC, ADCP, and CDC.

Exemplary modifications include but are not limited substitutions, insertions, and deletions at positions 234, 235, 236, 237, 267, 269, 325, 328, 330, and/or 331 (e.g., 330 and 331), wherein numbering is according to the EU index. Exemplary substitutions include but are not limited to 234A, 235E, 236R, 237A, 267R, 269R, 325L, 328R, 330S, and 331S (e.g., 330S, and 331S), wherein numbering is according to the EU index. An Fc variant can comprise 236R/328R. Other modifications for reducing FcγR and complement interactions include substitutions 297A, 234A, 235A, 237A, 318A, 228P, 236E, 268Q, 309L, 330S, 331S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691. For example, the human IgG1.3 Fc constant region contains L234A, L235E, and G237A substitutions. The IgG1fa.P238K (or IgG1.P238K) contains a P238K substitution. The IgG1.If comprises L234A, L235E, G237A, A330S, and P331S substitutions. (All numbering under the EU index.)

Fc variants that enhance affinity for an inhibitory receptor FcγRIIb can also be used. Such variants can provide an Fc fusion protein with immunomodulatory activities related to FcγRIIb cells, including for example, B cells and monocytes. In one embodiment, the Fc variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, 330, 331, and 332, according to the EU index. Exemplary substitutions for enhancing FcγRIIb affinity include but are not limited to 234A, 234D, 234E, 234F, 234W, 235D, 235E, 235F, 235R, 235Y, 236D, 236N, 237A, 237D, 237N, 239D, 239E, 266M, 267D, 267E, 268D, 268E, 327D, 327E, 328F, 328W, 328Y, 330S, 331S, and 332E. Exemplary substitutions include 235Y, 236D, 239D, 266M, 267E, 268D, 268E, 328F, 328W, and 328Y. Other Fc variants for enhancing binding to FcγRIIb include 235Y/267E, 236D/267E, 239D/268D, 239D/267E, 267E/268D, 267E/268E, and 267E/328F. (All numbering under the EU index.)

Other modifications for enhancing FcγR and complement interactions include but are not limited to substitutions 298 A, 333A, 334A, 326A, 2471, 339D, 339Q, 280H, 290S, 298D, 298V, 243L, 292P, 300L, 396L, 3051, and 396L. These and other modifications are reviewed in Strohl, 2009, Current Opinion in Biotechnology 20:685-691. Fc modifications that increase binding to an Fc7 receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in Patent Publication No. WO 00/42072.

Optionally, the Fc region can comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; PCX Patent Publications WO 00/42072; WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/0201 14).

The affinities and binding properties of an Fc region for its ligand can be determined by a variety of in vitro assay methods (biochemical or immunological based assays) known in the art including but not limited to, equilibrium methods (e.g., enzyme-linked immunosorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods can utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental immunology, 4th Ed., Lippincott-Raven, Philadelphia (1999), which focuses on antibody-immunogen interactions.

In certain embodiments, the antibody is modified to increase its biological half-life. Various approaches are possible. For example, this can be done by increasing the binding affinity of the Fc region for FcRn, For example, one or more of more of following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 2591, 308F, 428L, 428M, 434S, 4341 1. 434F, 434Y, and 434X1. Other variants that increase Fc binding to FcRn include: 250E, 250Q, 428 L, 428F, 250Q/428L (Hinton et al. 2004, J Biol. Chem. 279(8): 6213-6216, Hinton et al. 2006 Journal of Immunology 176:346-356), 256A, 272A, 286A, 305A, 307A, 307Q, 31 1A, 312A, 376A, 378Q, 380A, 382A, 434A (Shields et al., Journal of Biological Chemistry, 2001, 276(9):6591-6604), 252F, 252T, 252Y, 252W, 254T, 256S, 256R, 256Q, 256E, 256D, 256T, 309P, 31 1 S, 433R, 433S, 4331, 433P, 433Q, 434H, 434F, 434Y, 252Y/254T/256E, 433K/434F/436H, 308T/309P/311S (Dall'Acqua et al. Journal of Immunology, 2002, 169:5171-5180, Dal'Acqua et al., 2006, Journal of Biological Chemistry 281:23514-23524). Other modifications for modulating FcRn binding are described in Yeung et al., 2010, J Immunol, 182:7663-7671.

In certain embodiments, hybrid IgG isotypes with particular biological characteristics can be used. For example, an IgG1/IgG3 hybrid variant can be constructed by substituting IgG1 positions in the CH2 and/or CH3 region with the amino acids from IgG3 at positions where the two isotypes differ. Thus a hybrid variant IgG antibody can be constructed that comprises one or more substitutions, e.g., 274Q, 276K, 300F, 339T, 356E, 358M, 384S, 392N, 397M, 4221, 435R, and 436F. In some embodiments described herein, an IgG1/IgG2 hybrid variant can be constructed by substituting IgG2 positions in the CH2 and/or CH3 region with amino acids from IgG1 at positions where the two isotypes differ. Thus a hybrid variant IgG antibody can be constructed that comprises one or more substitutions, e.g., one or more of the following amino acid substitutions: 233E, 234L, 235L, +236G (referring to an insertion of a glycine at position 236), and 327A.

Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that can be used include: S298A/E333A/L334A, S239D/I332E, S239D/I332E/A330L, L235V/F243L/R292P/Y300L/P396L, and M428L/N434S.

In certain embodiments, an Fc is chosen that has reduced binding to FcγRs. An exemplary Fc, e.g., IgG1 Fc, with reduced FcγR binding comprises the following three amino acid substitutions: L234A, L235E and G237A.

In certain embodiments, an Fc is chosen that has reduced complement fixation. An exemplary Fc, e.g., IgG1 Fc, with reduced complement fixation has the following two amino acid substitutions: A330S and P331S.

In certain embodiments, an Fc is chosen that has essentially no effector function, i.e., it has reduced binding to FcγRs and reduced complement fixation. An exemplary Fc, e.g., IgG1 Fc, that is effectorless comprises the following five mutations: L234A, L235E, G237A, A330S and P331S.

When using an IgG4 constant domain, it can include the substitution S228P, which mimics the hinge sequence in IgG1 and thereby stabilizes IgG4 molecules. Fc modifications described in WO 2017/087678 or WO2016081746 may also be used.

In certain embodiments, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in further detail in U.S. Pat. Nos. 5,714,350 and 6,350,861 by Co et al.

Glycosylation of the constant region on N297 can be prevented by mutating the N297 residue to another residue, e.g., N297A, and/or by mutating an adjacent amino acid, e.g., 298 to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Led 3 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases {e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17: 176-180).

Another modification of the antibodies described herein is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. In some embodiments, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies described herein. See for example, EP 0 154 316 by Nishimura et al. and EP 0 401 384 by Ishikawa et al.

### IV. Nucleic Acid Molecules Encoding Anti-PSMA Antibodies

Nucleic acid molecules comprising polynucleotides that encode one or more chains of anti-PSMA antibodies described herein are provided. In some embodiments, a nucleic acid molecule comprises a polynucleotide that encodes a heavy chain or a light chain of an anti-PSMA antibody. In some embodiments, provided is an isolated nucleic acid encoding any one of the anti-PSMA antibodies provided herein. In some embodiments, a nucleic acid molecule comprises both a polynucleotide that encodes a heavy chain and a polynucleotide that encodes a light chain, of an anti-PSMA antibody. In some embodiments, a first nucleic acid molecule comprises a first polynucleotide that encodes a heavy chain and a second nucleic acid molecule comprises a second polynucleotide that encodes a light chain.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an anti-PSMA antibody comprises a nucleotide sequence that encodes a leader sequence, which, when translated, is located at the N terminus of the heavy chain or light chain. The leader sequence may be the native heavy or light chain leader sequence, or may be another heterologous leader sequence.

Nucleic acid molecules may be constructed using recombinant DNA techniques conventional in the art. In some embodiments, a nucleic acid molecule is an expression vector that is suitable for expression in a selected host cell.

### V. Anti-PSMA Antibody Expression and Production

### A. Vectors

Vectors comprising polynucleotides that encode anti-PSMA heavy chains and/or anti-PSMA light chains are provided. In some embodiments, provided herein is a vector comprising a nucleic acid encoding any one of the anti-PSMA antibodies described herein. Vectors comprising polynucleotides that encode anti-PSMA heavy chains and/or anti-PSMA light chains are also provided. Such vectors include, but are not limited to, DNA vectors, RNA vectors (e.g., mRNA and circular RNA, self-amplifying RNA vectors, etc.), phage vectors, viral vectors (e.g., pox virus vectors, vaccinia virus vectors, adenovirus vectors, modified vaccinia virus Ankara (MVA) vectors, etc.), retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004).

In some embodiments, a vector is chosen for in vivo expression anti-PSMA heavy chains and/or anti-PSMA light chains in animals, including humans. In some such embodiments, expression of the polypeptide is under the control of a promoter that functions in a tissue-specific manner. For example, liver-specific promoters are described, e.g., in PCT Publication No. WO 2006/076288.

### B. Host Cells

In various embodiments, anti-PSMA heavy chains and/or anti-PSMA light chains may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. In some embodiments, provided herein are host cells comprising an isolated nucleic acid encoding any one of the anti-PSMA antibodies described herein. In some embodiments, provided herein are host cells comprising a vector comprising a nucleic acid encoding any one of the anti-PSMA antibodies described herein. In some embodiments, provided herein is a host cell that produces any one of the anti-PSMA antibodies described herein. Exemplary eukaryotic cells that may be used to express polypeptides include, but are not limited to, COS cells, including COS 7 cells; 293 cells, including 293-6E cells; CHO cells, including CHO-S and DG44 cells; PER.C6® cells (Crucell); and NSO cells. In some embodiments, anti-PSMA heavy chains and/or anti-PSMA light chains may be expressed in yeast. See, e.g., U.S. Publication No. US 2006/0270045 A1. In some embodiments, a particular eukaryotic host cell is selected based on its ability to make desired post-translational modifications to the anti-PSMA heavy chains and/or anti-PSMA light chains. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

In some embodiments, one or more polypeptides may be produced in vivo in an animal that has been engineered or transfected with one or more nucleic acid molecules encoding the polypeptides, according to any suitable method.

In some embodiments, provided herein is a method for making an anti-PSMA antibody described herein, comprising culturing a host cell described herein under conditions suitable for expression of the antibody. In some embodiments, the method further comprises recovering the antibody produced by the host cell.

### C. Purification of Anti-PSMA Antibodies

Anti-PSMA antibodies may be purified by any suitable method. Such methods include, but are not limited to, the use of affinity matrices or hydrophobic interaction chromatography or size exclusion chromatography.

### D. Cell-Free Production of Anti-PSMA Antibodies

In some embodiments, an anti-PSMA antibody is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### VI. Exemplary Antibody-Drug Conjugates

The anti-PSMA antibodies described herein may be included in an antibody-drug conjugate. An antibody-drug conjugate typically comprises three components: Antibody, Payload, and Linker. In some embodiments, the Antibody targets the ADC to a particular cell type and may also elicit a therapeutic response, the Payload elicits a desired therapeutic response, and the Linker attaches the Payload to the Antibody. Often, the Payload is coupled via the Linker to an Antibody that specifically targets a certain tumor antigen (e.g., a protein that is expressed at higher levels on tumor cells compared to normal cells). In some embodiments, an antibody binds its antigen on the surface of a cell, such as a cancer cell, and the ADC is internalized by the cell. In some such embodiments, after the ADC is internalized, the Payload elicits the desired therapeutic response, for example, in the case of a Payload which is an anticancer agent, the Payload inhibits the expansion of, or kills, the cancer cell.

In some embodiments, the Payload is an anticancer drug. In some embodiments, the Payload is a radioisotope.

### VII. Therapeutic Compositions and Methods

### A. Methods of Treating Diseases

In one embodiment, provided herein is a method of treating and/or preventing cancer, which comprises administering to a patient a compound, antibody, or antibody-drug conjugate provided herein.

In another embodiment, provided herein is method of managing cancer, which comprises administering to a patient a compound, antibody, or antibody-drug conjugate provided herein.

Also provided herein are methods of treating patients who have been previously treated for cancer but are non-responsive to standard therapies, as well as those who have not previously been treated. Also encompassed are methods of treating patients regardless of patient's age, although some diseases or disorders are more common in certain age groups. Further encompassed are methods of treating patients who have undergone surgery in an attempt to treat the disease or condition at issue, as well as those who have not. Because patients with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

In some embodiments, methods of treating cancer are provided, wherein the cancer is prostate cancer, castration resistant prostate cancer (CRPC), or metastatic castration resistant prostate cancer (mCRPC).

In some embodiments, the cancer is a PSMA+ cancer. In some embodiments, the cancer is a PSMA+ solid cancer. In some embodiments, the PSMA+ solid cancer is prostate cancer, castration resistant prostate cancer (CRPC), or metastatic castration resistant prostate cancer (mCRPC).

In certain embodiments, the patient to be treated with one of the methods provided herein has not been treated with anticancer therapy prior to the administration of a compound, antibody, or antibody-drug conjugate provided herein. In certain embodiments, the patient to be treated with one of the methods provided herein has been treated with anticancer therapy prior to the administration of a compound, antibody, or antibody-drug conjugate provided herein. In certain embodiments, the patient to be treated with one of the methods provided herein has developed drug resistance to the anticancer therapy.

The methods provided herein encompass treating a patient regardless of patient's age, although some diseases or disorders are more common in certain age groups.

### B. Routes of Administration and Carriers

Provided herein are compositions (e.g., pharmaceutical compositions) comprising a compound, antibody, or antibody-drug conjugate provided herein and one or more pharmaceutically acceptable carriers.

In various embodiments, a compound, antibody, or antibody-drug conjugate provided herein may be administered in vivo by various routes, including, but not limited to, oral, intra-arterial, parenteral (including intravenous and subcutaneous), intranasal, intramuscular, intracardiac, intraventricular, intratracheal, buccal, rectal, intraperitoneal, intradermal, topical, transdermal, and intrathecal, or otherwise by implantation or inhalation. The subject compositions may be formulated into preparations, such as liquid formulations or formulations suitable for injections, inhalations, and the like. Alternatively, in some embodiments, the composition may be provided as a lyophilized powder that may be reconstituted upon addition of an appropriate liquid or carrier, for example, sterile water. The appropriate formulation and route of administration may be selected according to the intended application.

In various embodiments, compositions comprising a compound, antibody, or antibody-drug conjugate provided herein are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available.

### VII. Examples

### Example 1: Generation of Antibodies Binding to PSMA

Anti-PSMA monoclonal antibodies were generated by modifying a benchmark antibody by affinity maturation and removal of post-translational modifications. 3 monoclonal anti-PSMA antibodies (data not shown) were identified with desired attributes compared to the benchmark antibody, including high affinity to human PSMA, fast internalization, lower immunogenicity, cyno-cross reactivity, favorable developability, and potent (sub nM IC50) in piggyback Antibody Drug Conjugate (ADC) killing assays. However, experimental and in silico studies (data not shown) indicated that the 3 antibodies were too hydrophobic for further development.

Next, 24 antibodies were generated by changing the heavy chain CDR-2 and FR-2 regions of the starting benchmark antibody. Using Interactive Screening and Protein Reengineering Interface (ISPRI), the immunogenic potential of the generated antibodies was analysed with EpiVax®. Of the 24 antibodies that were generated by changing heavy chain CDR-2 and FR-2 regions, 17 antibodies did not have the desired hydrophobicity or immunogenicity.

Of the 24 antibodies, 7 antibodies exhibited improved hydrophobicity (lower hydrophobicity) and better immunogenicity EpiVax® scores. Binding kinetics of the antibodies were assessed by surface plasmon resonance (SPR). The 7 antibodies that exhibited lower hydrophobicity were also found to have high affinity to human PSMA by SPR. The cell binding profiles (IC50) of those 7 antibodies were also similar to the binding profile exhibited by one of the 3 parental antibodies. The 7 antibodies also exhibited great improvement in conjugation yields by decreasing ADC aggregation.

Next, anti-PSMA antibody-GSPT1 degrader drug conjugates of these 7 antibodies were prepared by incubating a payload GSPT1-degrader reagent with the 7 antibodies in suitable conditions and then purifying the conjugates. The resulting ADCs were then administered to cells and cell viability was determined. The 7 GSPT1-conjugated ADCs showed pM potency and exhibited IC50 values that were similar to the benchmark antibody. Overall, the 7 antibodies exhibited high affinities for human PSMA, rapid internalization rates, cross reactivity to cynomolgus monkey PSMA, favorable developability, and potent (sub nM IC50) in piggyback ADC killing assays (data not shown).

Binding and internalization into human corneal epithelial cells (HCEC) serve as a proxy for ADC toxicity and internalization into HCEC correlates with hydrophobicity (HIC RT). Accordingly, the 7 antibodies were next tested in HCEC cells. The 7 antibodies demonstrated moderate binding and internalization into HCEC, which suggested a need for further improvement.

Of the 7 antibodies, 4 antibodies showed improved hydrophobicity, binding kinetics (K_{D} as measured by SPR), and ADC-mediated cytotoxicity on PSMA-positive cells compared to the other 3 antibodies (data not shown). One of the 4 antibodies, anti-PSMA-Ab2 antibody, was therefore selected for further optimization based on differences in hydrophobicity, binding K_{D} (SPR) and ADC cytotoxicity on PSMA positive cells.

Anti-PSMA-Ab2 demonstrated improved mouse (NSG) in vivo pharmacokinetics. The maximum concentration (Cmax), time maximum concentration reached (Tmax), area under the curve at the last time point (AUCₗₐₛₜ), clearance rate (Cl), steady-state volume of distribution (Vss), and half-life (t_{1/2}) were measured. The results are shown in Table E1. Surprisingly, anti-PSMA-Ab2 showed a t_{1/2} of 10 days as compared to the benchmark antibody (t_{1/2} of 2.7 days). Thus, anti-PSMA-Ab2 had a three times slower clearance rate and a half-life compared to the benchmark antibody, as shown in Table E1 below.

TABLE E1
Mean PK parameters

| | Benchmark | Ab2 |
|---|---|---|
| AUCₗₐₛₜ | 111354 | 147514 |
| CL (mL/day/kg) | 30.2 | 10.6 |
| Vss (mL/kg) | 105 | 154 |
| t_{1/2} (days) | 2.7 | 10.4 |

Anti-PSMA-Ab2, however, demonstrated moderate non-specific internalization into human corneal epithelial cells (HCEC) that was abrogated by adding a micropinocytosis inhibitor (EIPA). This indicated that the internalization of anti-PSMA-Ab2 was non-specific. Abberant positive charge/charge patches and/or aberrant hydrophobicity can drive polyreactivity and non-specificity leading to non-specific cellular uptake and reduced pharmacokinetics.

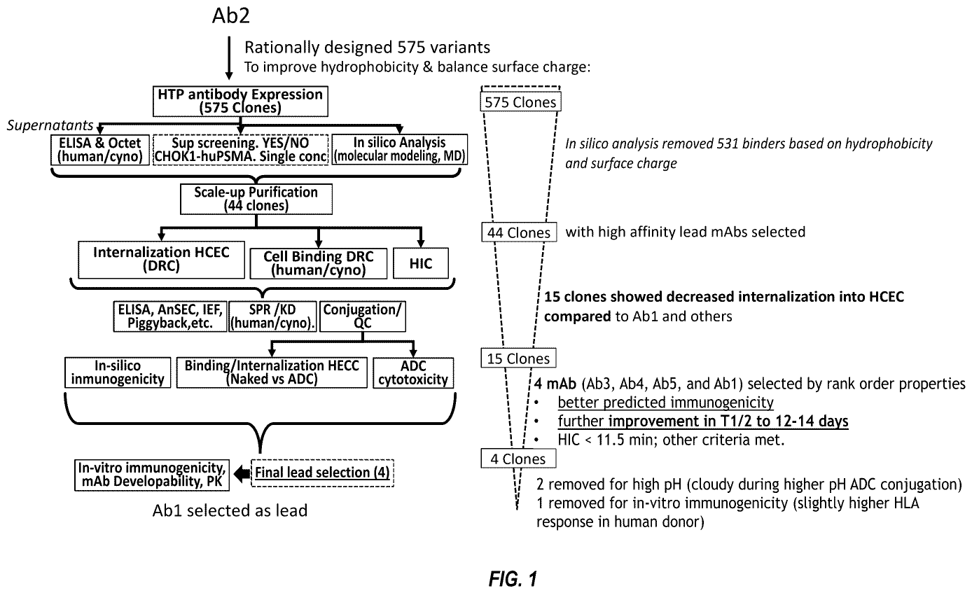

To further improve hydrophobicity and to balance the surface charge, anti-PSMA-Ab2 was modified by substituting: (i) 4 positions in the heavy chain at HCDR1, HCDR2, and HCDR3 with 6 amino acids; (ii) 3 positions in the light chain in LCDR2 and LCDR3 with 4 amino acids, and (iii) 2 positions in the light chain framework region with 2 amino acids. These substitutions resulted in 575 rationally designed variants. The 575 variants were generated using high-throughput (HTP) antibody expression processes. As shown in FIG. 1, the HTP expression process was combined with ELISA and Octet assays, CHOK1-huPSMA expression systems, and in silico analysis, including molecular modelling and low-mode molecular dynamics. As shown in FIG. 1, 531 antibodies were eliminated based on hydrophobicity and surface charge during the in silico analysis. The remaining 44 antibodies demonstrated high affinity and were selected for scale up purification and further testing.

### Characterization of the 44 Selected Variants of Anti-PSMA-Ab2

The binding profiles of the 44 selected variants were assessed as shown in FIG. 1. Most variants showed comparable binding profiles to human and cynomolgus (cyno) PSMA compared to the parental lineage (anti-PSMA-Ab2). This indicated that the amino acid substitutions did not negatively affect binding to human PSMA. The differences in IC50 between human and cynomolgus PSMA were 1-4 fold for all variants tested. Internalization of the 44 selected variants into HCEC cells was also assessed. Of the 44 selected variants, 15 variants showed improved (i.e., decreased) internalization into HCEC cells compared to the anti-PSMA-Ab2 and the benchmark antibody, and were selected for further characterization (FIG. 1).

The 15 variants were further characterized for biophysical properties using Analytical Size Exclusion Chromatography (AnSEC), isoelectric focusing (IEF), mass photometry, and analytical hydrophobic interaction chromatography (HIC). GSPT1-conjugated ADCs were generated from the 15 antibodies, and purified, resulting in high yields of approximately 60-93%. The efficacies of the ADCs were analysed by measuring the Drug to Antibody Ratio (DAR) using Liquid Chromatography/Mass Spectrometry (LCMS). DALR refers to the average number of drugs linked to each antibody and is a measure of ADC efficacy. Binding kinetics were assessed by surface plasmon resonance (SPR). Cytotoxicity was evaluated in LNCaP cells. No cytotoxicity in PSMA negative LNCaP cells was observed.

Of the 15 variants, 4 variants showed improved EpiVax immunogenicity scores and t_{1/2} (12-14 days as compared to the benchmark which had 7 days in nude mice). The 4 variants demonstrated low risk for in silco immunogenicity as compared to the benchmark and the anti-PSMA-Ab2 antibodies (risk level from highest to lowest: benchmark>Ab2>Ab1/Ab5/Ab4/Ab3).

The 4 variants contained the same T cell epitope in the VH CDR1. This T cell epitope has a medium HLA binding potential. This T cell epitope is unlikely to be processed and presented on MHC class II by antigen presenting cells, and shares homology with human IgG. A dendritic cell-peripheral blood mononuclear cell (DC-PBMC) assay was performed to evaluate the potential in vitro immunogenicity of the variants. Anti-PSMA-Ab1 and two other antibodies generated less than 200 positive responses suggesting a low risk for immunogenicity. Lead antibodies had lower Epivax scores and good toxicity results. Table E2 below shows the results.

TABLE E2

| Key Criteria | Anti-PSMA-Ab3 | Anti-PSMA-Ab4 | Anti-PSMA-Ab5 | Anti-PSMA-Ab1 |
|---|---|---|---|---|
| Favorable PK | Improved, | Improved, | Improved, | Improved, |
| (Benchmark T_{1/2} | ½ life 14.1 days | ½ life 12.5 days | ½ life 14.2 days | ½ life 12.9 days |
| 9.5 days) | | | | |
| In-vitro | 10% | >20% | ~12% | ~15% |
| immunogenicity | | | | |
| (% Positive donors, | | | | |
| Threshold 20%) | | | | |
| mAb Developability | HIC < 11.5 min; | HIC < 11.5 min; | HIC < 11.5 min; | HIC < 11.5 min; |
| HIC | other criteria met. | other criteria met. | other criteria met. | other criteria met. |
| pI, pH hold | pI 7.1, | pI 7.9, | pI 7.9, | pI 7.9, |
| Chemical | pH 6 hold OK | pH 6 hold OK | pH 6 hold OK | pH 6 hold OK |
| Modifications | pH 8.3 hold. | pH 8.3 hold OK. | pH 8.3 Hold, | pH 8.3 hold OK. |
| | Cloudy at 4C. | | NOT OK. | |
| | Oxidation CQA. W50/CDR2 (binding fingerprint). | | | |
| | Control strategy in place by DPAS (light protection & DPTA in formulation) | | | |
| ADC format | Similar IC50 across mAb | | | |
| Cytotoxicity | Similar Serum stability across lead PSMA mAb. | | | |
| Serum stability | Improved Serum stability compared to Benchmark PSMA mAb. | | | |
| Bystander Effect | Similar Bystander Kill across mAb | | | |
| In vivo efficacy @ | 89-94% TVR | 89-92% TVR | 86-90% TVR | 90-90.1% TVR |
| single-dose | | | | |
| OMP-PR22 PDX, | | | | |
| mCRPC | | | | |

### Example 2: Anti-PSMA Antibody Affinity for PSMA of Various Species

Binding kinetics of antibody PSMA-Ab1 were assessed by surface plasmon resonance (SPR). The association rate (ka), dissociation rate (kd) and affinity (K_{D}) of Ab1 for human, cynomolgus monkey, dog, rat, and mouse PSMA are shown in Table E3.

TABLE E3
Binding of Ab1 to PSMA from various species

| Antibody | Analyte | ka (1/Ms) | kd (1/s) | KD (nM) |
|---|---|---|---|---|
| Ab1 | Human PSMA | 2.81E+05 | 8.97E−04 | 3.2 |
| | Cyno PSMA | 2.00E+05 | 9.27E−04 | 4.6 |
| | Dog PSMA | 1.20E+05 | 1.11E−02 | 92.2 |

| | |
|---|---|
| Rat PSMA | No Binding |
| Mouse PSMA | No Binding |

### Example 3: In Vivo Pharmacokinetics of Anti-PSMA Antibody

Non-tumor bearing mice were administered a single IV bolus injection of 10 mg/kg Ab1, Ab3, Ab4, Ab, or the benchmark anti-PSMA antibody. The maximum concentration (Cmax), time maximum concentration reached (Tmax), area under the curve at the last time point (AUCₗₐₛₜ), clearance rate (Cl), steady-state volume of distribution (Vss), and half-life (T_{1/2}) were measured. The results are shown in Table E4. All four anti-PSMA antibodies showed improved pharmacokinetics over the benchmark antibody.

TABLE E4
Pharmacokinetic parameters for anti-PSMA antibodies
Ab1, Ab3, Ab4, Ab5, and benchmark antibody

| | Compound | Benchmark | Ab1 | Ab3 | Ab4 | Ab5 |
|---|---|---|---|---|---|---|
| | Analyte | total_Ab | total_Ab | total_Ab | total_Ab | total_Ab |
| plasma | Cmax (μM) | 0.98 | 1.16 | 1.32 | 1.41 | 1.54 |
| | Tmax (h) | 6 | 6 | 6 | 6 | 6 |
| | AUClast (h*μM) | 135.18 | 218.48 | 225.94 | 224.65 | 209.4 |
| | Cl (mL/day/kg) | 9.1 | 4.8 | 4.5 | 4.7 | 4.7 |
| | Vss (mL/kg) | 118.0 | 87.7 | 88.2 | 83.5 | 94.3 |
| | T ½ (day) | 9.5 | 12.9 | 14.1 | 12.5 | 14.2 |

### Example 4: Selection of anti-PSMA Antibody with Superior Properties

The in vitro immunogenicity of the anti-PSMA antibodies Ab1, Ab3, Ab4, and Ab5 was further assessed using human donor blood samples. Ab4 showed a human leukocyte antigen (HLA) response that was slightly higher than the 200 threshold. Ab3 and Ab5 were cloudy during ADC conjugation at higher pH levels (pH of 8.3).

Overall, the ani-PSMA Ab1 antibody showed superior results overall compared to the benchmark antibody, including binding affinity to human PSMA, selectivity for human PSMA (i.e., low off-target binding), species cross-reactivity (e.g., high affinity for human and cyno PSMA without high affinity for PSMA of other mammalian species), developability (e.g., stability in formulation), immunogenicity, pharmacokinetics, and in vivo biodistribution. These superior results are summarized in Table E5.

TABLE E5
Summary of results

| | Attribute/Assay | | |
|---|---|---|---|
| Category | Description | Target Values | Anti-PSMA-Ab1 |
| Binding, | Human affinity (SPR) | Range 1-30 nM | K_{D} = 3.2 nM |
| selectivity, and | Tox species affinity | Within 10-fold of | Cyno K_{D} = 4.6 nM |
| species cross- | | human PSMA | Dog K_{D} ~30x human |
| reactivity | Poly-reactivity | Favorable profile in | Meets criteria |
| | | BVP, ECM, NPAL | |
| | | assessment | |
| | Poly selectivity | No or derisked off- | Meets criteria |
| | (Retrogenix) | target | |
| | Epitope | Target membrane | Membrane distal domain |
| | | PSMA isoforms | |
| | Fc backbone | Optimal safety | hIgG1, Fc WT, f Allotype |
| | | without impact to | |
| | | potency | |
| | Off-target dependent | Minimal uptake by | Meets criteria |
| | uptake | HCEC, hepatocytes | |
| | | & Kupffer cells | |
| | | Minimal cytokine | |
| | | release in human | |
| | | WB assay at ≤ 1 μM | |
| Developability | Aggregation propensity | No significant | HIC < 11.5 min, pI 7.9, |
| (DSi stability and | and chemical | soluble aggregate | pH 6 and pH 8.3 hold OK, |
| formulation fit) | modification liability | formation/amino | W50 oxidation LC-CDR2 |
| | assessment (1-month | acid modifications | identified, mitigation plan in |
| | stability) | post-stress | place |
| Immunogenicity | In vitro | % Positive | 15% |
| | immunogenicity | donors < 20% | |
| PK | Exposure profile | Mouse PK better | T_{1/2} = 12.9 days |
| | supporting single dose | than benchmark | |
| | efficacy in nude mice | (T_{1/2} 9.5 days) | |
| | model | | |
| In vivo | Cyno and mouse | Normal mAb tissue | Meets criteria |
| Biodistribution | imaging | distribution in cyno | |
| | | Target organ | |
| | | distribution in | |
| | | tumor bearing mice | |

### Example 5: In Vivo Efficacy of PSMA-GSPT1 ADC

This Example describes the in vivo efficacy of PSMA-GSPT1 ADCs in an OMP-PR22 patient-derived xenograft (PDX) mouse model. Nude mice (N=25; n=5 per group) were inoculated with 1 million OMP-PR22 PDX cells (Day 0). The mice exhibited 80-90% PSMA positive cells and developed tumors measuring approximately 100-200 mm3 in size on Day 34 post-inoculation. On Day 34, the mice were administered either a vehicle or a single IV bolus injection of 3 mg/kg anti-PSMA-Ab1, Ab3, Ab4, or Ab5 conjugated to a GSPT1 payload. The efficacy endpoints included tumor volume reduction and body weight changes.

Antitumor efficacies were assessed using a tumor growth delay (TGD) metric. Tumor volume was measured twice weekly and the time required for each tumor to reach a predefined endpoint volume was determined. The median time-to-endpoint (TTE) was calculated for each group. Absolute TGD was defined as the difference between treated and control groups (T−C). Percent tumor growth delay (% TGD) was calculated as [(T−C)/C]×100, where T and C represent the median TTE of treated and vehicle control groups, respectively. TGD was calculated on the last day (Day 85) of measurement for vehicle group. Tumors in mice treated with the anti-PSMA ADCs were measured until Day 152.

The results are shown in Table E6. A single IV dose of 3 mg/kg Ab1 or Ab3 led to significant inhibition of tumor growth.

TABLE E6
In vivo efficacy of PSMA-GSPT1 ADCs in an OMP-PR22 PDX mouse model.

| | | % Tumor | | | |
|---|---|---|---|---|---|
| | | Volume | | | % Tumor |
| | Dose | Reduction | | | growth delay |
| | (mg/kg) | (TVR) | % Regression | | (TGD)/ |
| ADC | IV, bolus | (Day 85) | (Day 85) | p value | latency |
| Vehicle | 0 | — | — | — | — |
| Anti-PSMA-Ab3 | 3 | 88.92 | 21.06 | 0.0008 | 444.31 |
| GSPT1 | | | | | |
| Anti-PSMA-Ab4 | 3 | 87.16 | 26.96 | 0.0014 | 207.31 |
| GSPT1 | | | | | |
| Anti-PSMA-Ab5 | 3 | 86.12 | 10.61 | 0.0012 | 132.29 |
| GSPT1 | | | | | |
| Anti-PSMA-Ab1 | 3 | 90.86 | 34.38 | 0.0011 | 193.25 |
| GSPT1 | | | | | |

IX. TABLE OF CERTAIN SEQUENCES
The following table provides a listing of certain sequences
referenced herein.

| | SEQ | |
|---|---|---|
| Description | ID | Sequence |
| PSMA-Ab1 | 1 | EVQLVQSGPEVKKPGATVKISCKTSGYTFTEYTIHWVRQAPGKGLEWIGNINPREGG |
| heavy chain | | TTYNQKFEDKATLTVDKSTDTAYMELSSLRSEDTAVYYCAAGWNFDSWGQGTLLTVS |
| variable | | S |
| region (VH) | | |
| PSMA-Ab1 | 2 | DIQMTQSPSSLSTSVGDRVTLTCKASQDIGTAVDWYQQKPGTSPKLLIYWASTRHTG |
| light chain | | IPSRFSGSGSGTDFTLTISSLQPEDAADYYCQQYSSYPLTFGPGTKVEIK |
| variable | | |
| region (VL) | | |
| PSMA-Ab1 | 3 | EYTIH |
| HCDR1 | | |
| (Kabat) | | |
| PSMA-Ab1 | 4 | GYTFTEY |
| HCDR1 | | |
| (Chothia, | | |
| IMGT) | | |
| PSMA-Ab1 | 5 | GYTFTEYTIH |
| HCDR1 | | |
| (AbM) | | |
| PSMA-Ab1 | 6 | NINPREGGTTYNQKFED |
| HCDR2 | | |
| (Kabat) | | |
| PSMA-Ab1 | 7 | NPREGG |
| HCDR2 | | |
| (Chothia) | | |
| PSMA-Ab1 | 8 | INPREGGT |
| HCDR2 | | |
| (IMGT) | | |
| PSMA-Ab1 | 9 | NINPREGGTT |
| HCDR2 | | |
| (AbM) | | |
| PSMA-Ab1 | 10 | GWNFDS |
| HCDR3 | | |
| (Kabat, | | |
| Chothia, | | |
| AbM) | | |
| PSMA-Ab1 | 11 | AAGWNFDS |
| HCDR3 | | |
| (IMGT) | | |
| PSMA-Ab1 | 12 | KASQDIGTAVD |
| LCDR1 | | |
| (Kabat, | | |
| Chothia, | | |
| AbM) | | |
| PSMA-Ab1 | 13 | QDIGTA |
| LCDR1 | | |
| (IMGT) | | |
| PSMA-Ab1 | 14 | WASTRHT |
| LCDR2 | | |
| (Kabat, | | |
| Chothia, | | |
| AbM) | | |
| PSMA-Ab1 | 15 | WAS |
| LCDR2 | | |
| (IMGT) | | |
| PSMA-Ab1 | 16 | QQYSSYPLT |
| LCDR3 | | |
| (Kabat, | | |
| Chothia, | | |
| IMGT, AbM) | | |
| PSMA-Ab1 | 17 | EVQLVQSGPEVKKPGATVKISCKTSGYTFTEYTIHWVRQAPGKGLEWIGNINPREGG |
| heavy chain | | TTYNQKFEDKATLTVDKSTDTAYMELSSLRSEDTAVYYCAAGWNFDSWGQGTLLTVS |
| (HC) | | SASTKGPSVFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKSCDKTHTCPPCP |
| | | APELLGGPSVFLFPPKPKDTLMISRTPEVTCVVVDVSHEDPEVKENWYVDGVEVHNA |
| | | KTKPREEQYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |
| PSMA-Ab1 | 18 | DIQMTQSPSSLSTSVGDRVTLTCKASQDIGTAVDWYQQKPGTSPKLLIYWASTRHTG |
| light chain | | IPSRFSGSGSGTDFTLTISSLQPEDAADYYCQQYSSYPLTFGPGTKVEIKRTVAAPS |
| (LC) | | VFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSENRGEC |
| Heavy chain | 19 | ASTKGPSVFPLAPSSKSTSGGTAALGCLVKDYFPEPVTVSWNSGALTSGVHTFPAVL |
| constant | | QSSGLYSLSSVVTVPSSSLGTQTYICNVNHKPSNTKVDKRVEPKSCDKTHTCPPCPA |
| region (CH) | | PELLGGPSVFLFPPKPKDTLMISRTPEVTCVVVDVSHEDPEVKENWYVDGVEVHNAK |
| | | TKPREEQYNSTYRVVSVLTVLHQDWLNGKEYKCKVSNKALPAPIEKTISKAKGQPRE |
| | | PQVYTLPPSREEMTKNQVSLTCLVKGFYPSDIAVEWESNGQPENNYKTTPPVLDSDG |
| | | SFFLYSKLTVDKSRWQQGNVFSCSVMHEALHNHYTQKSLSLSPG |
| Light chain | 20 | RTVAAPSVFIFPPSDEQLKSGTASVVCLLNNFYPREAKVQWKVDNALQSGNSQESVT |
| constant | | EQDSKDSTYSLSSTLTLSKADYEKHKVYACEVTHQGLSSPVTKSENRGEC |
| region (CL) | | |
| Human PSMA | 21 | MWNLLHETDSAVATARRPRWLCAGALVLAGGFFLLGFLFGWFIKSSNEATNITPKHN |
| (UniProt ID: | | MKAFLDELKAENIKKFLYNFTQIPHLAGTEQNFQLAKQIQSQWKEFGLDSVELAHYD |
| Q04609.1) | | VLLSYPNKTHPNYISIINEDGNEIFNTSLFEPPPPGYENVSDIVPPFSAFSPQGMPE |
| | | GDLVYVNYARTEDFFKLERDMKINCSGKIVIARYGKVFRGNKVKNAQLAGAKGVILY |
| | | SDPADYFAPGVKSYPDGWNLPGGGVQRGNILNLNGAGDPLTPGYPANEYAYRRGIAE |
| | | AVGLPSIPVHPIGYYDAQKLLEKMGGSAPPDSSWRGSLKVPYNVGPGFTGNFSTQKV |
| | | KMHIHSTNEVTRIYNVIGTLRGAVEPDRYVILGGHRDSWVFGGIDPQSGAAVVHEIV |
| | | RSFGTLKKEGWRPRRTILFASWDAEEFGLLGSTEWAEENSRLLQERGVAYINADSSI |
| | | EGNYTLRVDCTPLMYSLVHNLTKELKSPDEGFEGKSLYESWTKKSPSPEFSGMPRIS |
| | | KLGSGNDFEVFFQRLGIASGRARYTKNWETNKFSGYPLYHSVYETYELVEKFYDPMF |
| | | KYHLTVAQVRGGMVFELANSIVLPFDCRDYAVVLRKYADKIYSISMKHPQEMKTYSV |
| | | SFDSLFSAVKNFTEIASKFSERLQDFDKSNPIVLRMMNDQLMFLERAFIDPLGLPDR |
| | | PFYRHVIYAPSSHNKYAGESFPGIYDALFDIESKVDPSKAWGEVKRQIYVAAFTVQA |
| | | AAETLSEVA |
| Human PSMA | 26 | KSSNEATNITPKHNMKAFLDELKAENIKKFLYNFTQIPHLAGTEQNFQLAKQIQSQW |
| (lacking | | KEFGLDSVELAHYDVLLSYPNKTHPNYISIINEDGNEIFNTSLFEPPPPGYENVSDI |
| amino acids 1- | | VPPFSAFSPQGMPEGDLVYVNYARTEDFFKLERDMKINCSGKIVIARYGKVFRGNKV |
| 43) | | KNAQLAGAKGVILYSDPADYFAPGVKSYPDGWNLPGGGVQRGNILNLNGAGDPLTPG |
| | | YPANEYAYRRGIAEAVGLPSIPVHPIGYYDAQKLLEKMGGSAPPDSSWRGSLKVPYN |
| | | VGPGFTGNFSTQKVKMHIHSTNEVTRIYNVIGTLRGAVEPDRYVILGGHRDSWVFGG |
| | | IDPQSGAAVVHEIVRSFGTLKKEGWRPRRTILFASWDAEEFGLLGSTEWAEENSRLL |
| | | QERGVAYINADSSIEGNYTLRVDCTPLMYSLVHNLTKELKSPDEGFEGKSLYESWTK |
| | | KSPSPEFSGMPRISKLGSGNDFEVFFQRLGIASGRARYTKNWETNKFSGYPLYHSVY |
| | | ETYELVEKFYDPMFKYHLTVAQVRGGMVFELANSIVLPFDCRDYAVVLRKYADKIYS |
| | | ISMKHPQEMKTYSVSFDSLFSAVKNFTEIASKFSERLQDFDKSNPIVLRMMNDQLMF |
| | | LERAFIDPLGLPDRPFYRHVIYAPSSHNKYAGESFPGIYDALFDIESKVDPSKAWGE |
| | | VKRQIYVAAFTVQAAAETLSEVA |
| Cynomolgus | 22 | KSSSEATNITPKHNMKAFLDELKAENIKKFLHNFTQIPHLAGTEQNFQLAKQIQSQW |
| monkey | | KEFGLDSVELTHYDVLLSYPNKTHPNYISIINEDGNEIENTSLFEPPPAGYENVSDI |
| PSMA | | VPPFSAFSPQGMPEGDLVYVNYARTEDFFKLERDMKINCSGKIVIARYGKVFRGNKV |
| | | KNAQLAGATGVILYSDPADYFAPGVKSYPDGWNLPGGGVQRGNILNLNGAGDPLTPG |
| | | YPANEYAYRRGIAEAVGLPSIPVHPIGYYDAQKLLEKMGGSASPDSSWRGSLKVPYN |
| | | VGPGFTGNFSTQKVKMHIHSTSEVTRIYNVIGTLRGAVEPDRYVILGGHRDSWVFGG |
| | | IDPQSGAAVVHEIVRSFGTLKKEGWRPRRTILFASWDAEEFGLLGSTEWAEENSRLL |
| | | QERGVAYINADSSIEGNYTLRVDCTPLMYSLVYNLTKELESPDEGFEGKSLYESWTK |
| | | KSPSPEFSGMPRISKLGSGNDFEVFFQRLGIASGRARYTKNWETNKFSSYPLYHSVY |
| | | ETYELVEKFYDPMFKYHLTVAQVRGGMVFELANSVVLPFDCRDYAVVLRKYADKIYN |
| | | ISMKHPQEMKTYSVSFDSLFSAVKNFTEIASKFSERLRDEDKSNPILLRMMNDQLMF |
| | | LERAFIDPLGLPDRPFYRHVIYAPSSHNKYAGESFPGIYDALFDIESKVDPSQAWGE |
| | | VKRQISIATFTVQAAAETLSEVA |
| Canine PSMA | 23 | KSPSEPTNIVPQHDMKKAFLDELKAENIKRFLYNFTRLPHLAGTERNFQLAKQIQSQ |
| | | WKEFGLDSVELAHYDVLLSYPNKTHPNYISIIDEDGNEIFNSSLFEPPPPGYENVSD |
| | | VVPPFNAFSPQGMPEGDLVYVNYAQTEDFFKLERDLKINCSGKIVIARYGKIFRGNK |
| | | VKNAQLAGAKGVILYSDPADYFAPGVKSYPDGWNLPGGGVQRGNILNLNGAGDPLTP |
| | | GYPANEYAYRREVTEAVGLPSIPVHPIGYNDAQKLLEKMGGAAPPDSSWRGGLQVPY |
| | | NVGPGFTGNSSTQKVKMHIHSNNKVTRIYNVIGTLRGAVEPDRYVILGGHRDSWVFG |
| | | GIDPQSGAAVVHEIVRSFGTLKKEGWRPRRTILFASWDAEEFGLLGSTEWAEENSRV |
| | | LQERGVAYINADSSIEGNYTLRVDCTPLMYSLVYNLTKELQSPDEGFEGKSLYESWN |
| | | EKSPSPEFSGMPRISKLGSGNDFEVFFQRLGISSGRARYTKNWETSKFRSYPLYHSV |
| | | YETYELVEKFYDPTFKYHLTVAQVRGGMVFELANSIVLPFDCRDYAVVLRKYADKIY |
| | | NISMKHPQEMKTYSVSFDSLFSAVKNFTEIASKFNERLEDLDKKNPILLRIMNDQLM |
| | | FLERAFIDPLGLPDRPFYRHIIYAPSSHNKYAGESFPGIYDALFDIESKVDPSKAWG |
| | | EVKRQISIAAFTVQAAAGTLREVA |
| Rat PSMA | 24 | KPSNDSTSSVSYPGMKKAFLQELKAENIKKFLYNFTRTPHLAGTQHNFELAKQIHAQ |
| | | WKEFGLDLVELSDYDVLLSYPNKTHPNYISIINEDGNEIFKTSLAELSPPGYENISD |
| | | VVPPYSAFSPQGTPEGDLVYVNYARTEDFFKLERVMKINCSGKIVIARYGQVFRGNK |
| | | VKNAQLAGAKGIILYSDPADYFVPGVKSYPDGWNLPGGGVQRGNVLNLNGAGDPLTP |
| | | GYPANEYAYRHEFTEAVGLPSIPVHPIGYDDAQKLLEHMGGSAPPDSSWKGGLKVPY |
| | | NVGPGFAGNFSKQKVKLHIHSYNKVTRIYNVIGTLKGAVEPDRYVILGGHRDAWVFG |
| | | GIDPQSGAAVVHEIVRTFGTLKKKGWRPRRTILFASWDAEEFGLLGSTEWAEEHSRL |
| | | LQERGVAYINADSSIEGNYTLRVDCTPLMHSLVYNLTKELPSPDEGFEGKSLYDSWK |
| | | EKSPSTEFIGMPRISKLGSGNDFEVFFQRLGIASGRARYTKNWKNNKVSSYPLYHSV |
| | | YETYELVEKFYDPTFKYHLTVAQVRGAMVFELANSIVLPFDCQSYAVALKKHAETIY |
| | | NISMNHPQEMKAYMISFDSLFSAVNNFTDVASKFNQRLQDLDKSNPILLRILNDQLM |
| | | YLERAFIDPLGLPGRPFYRHIIYAPSSHNKYAGESFPGIYDALFDINNKVDTSKAWR |
| | | EVKRQISIAAFTVQAAAETLREVD |
| Mouse PSMA | 25 | IKPSNEATGNVSHSGMKKEFLHELKAENIKKFLYNFTRTPHLAGTQNNFELAKQIHD |
| | | QWKEFGLDLVELSHYDVLLSYPNKTHPNYISIINEDGNEIFKTSLSEQPPPGYENIS |
| | | DVVPPYSAFSPQGTPEGDLVYVNYARTEDFFKLEREMKISCSGKIVIARYGKVFRGN |
| | | MVKNAQLAGAKGMILYSDPADYFVPAVKSYPDGWNLPGGGVQRGNVLNLNGAGDPLT |
| | | PGYPANEHAYRHELTNAVGLPSIPVHPIGYDDAQKLLEHMGGPAPPDSSWKGGLKVP |
| | | YNVGPGFAGNFSTQKVKMHIHSYTKVTRIYNVIGTLKGALEPDRYVILGGHRDAWVE |
| | | GGIDPQSGAAVVHEIVRSFGTLKKKGRRPRRTILFASWDAEEFGLLGSTEWAEEHSR |
| | | LLQERGVAYINADSSIEGNYTLRVDCTPLMYSLVYNLTKELQSPDEGFEGKSLYDSW |
| | | KEKSPSPEFIGMPRISKLGSGNDFEVFFQRLGIASGRARYTKNWKTNKVSSYPLYHS |
| | | VYETYELVVKFYDPTFKYHLTVAQVRGAMVFELANSIVLPFDCQSYAVALKKYADTI |
| | | YNISMKHPQEMKAYMISFDSLFSAVNNFTDVASKFNQRLQELDKSNPILLRIMNDQL |
| | | MYLERAFIDPLGLPGRPFYRHIIYAPSSHNKYAGESFPGIYDALFDISSKVNASKAW |
| | | NEVKRQISIATFTVQAAAETLREVA |
| Anti-PSMA- | 27 | EVQLVQSGPEVKKPGATVKISCKTSGYTFTTYTIHWVRQAPGKGLEWIGNINPETGG |
| Ab3 | | TTYNQKFEDKATLTVDKSTDTAYMELSSLRSEDTAVYYCAAGWNFDYWGQGTLLTVS |
| heavy chain | | S |
| variable | | |
| region (VH) | | |
| Anti-PSMA- | 28 | DIQMTQSPSSLSTSVGDRVTLTCKASQDIGTAVDWYQQKPGKSPKLLIYWASTTHEG |
| Ab3 | | IPSRFSGSGSGTDFTLTISSLQPEDAADYYCQQYSSYPLTFGPGTKVEIK |
| light chain | | |
| variable | | |
| region (VL) | | |
| Anti-PSMA- | 29 | TYTIH |
| Ab3 | | |
| HCDR1 | | |
| (Kabat) | | |
| Anti-PSMA- | 30 | GYTFTTY |
| Ab3 | | |
| HCDR1 | | |
| (Chothia) | | |
| Anti-PSMA- | 31 | GYTFTTYT |
| Ab3 | | |
| HCDR1 | | |
| (IMGT) | | |
| Anti-PSMA- | 32 | GYTFTTYTIH |
| Ab3 | | |
| HCDR1 | | |
| (AbM) | | |
| Anti-PSMA- | 33 | NINPETGGTTYNQKFED |
| Ab3 | | |
| HCDR2 | | |
| (Kabat) | | |
| Anti-PSMA- | 34 | NPETGG |
| Ab3 | | |
| HCDR2 | | |
| (Chothia) | | |
| Anti-PSMA- | 35 | INPETGGT |
| Ab3 | | |
| HCDR2 | | |
| (IMGT) | | |
| PSMA-Ab3 | 36 | NINPETGGTT |
| HCDR2 | | |
| (AbM) | | |
| Anti-PSMA- | 37 | GWNFDY |
| Ab3 | | |
| HCDR3 | | |
| (Kabat, | | |
| Chothia, | | |
| AbM) | | |
| Anti-PSMA- | 38 | AAGWNFDY |
| Ab3 | | |
| HCDR3 | | |
| (IMGT) | | |
| PSMA-Ab3 | 39 | KASQDIGTAVD |
| LCDR1 | | |
| (Kabat, | | |
| Chothia, | | |
| AbM) | | |
| PSMA-Ab3 | 40 | QDIGTA |
| LCDR1 | | |
| (IMGT) | | |
| PSMA-Ab3 | 41 | WASTTHE |
| LCDR2 | | |
| (Kabat, | | |
| Chothia, | | |
| AbM) | | |
| PSMA-Ab3 | 42 | WAS |
| LCDR2 | | |
| (IMGT) | | |
| PSMA-Ab3 | 43 | QQYSSYPLT |
| LCDR3 | | |
| (Kabat, | | |
| Chothia, | | |
| IMGT, AbM) | | |
| PSMA-Ab3 | 44 | EVQLVQSGPEVKKPGATVKISCKTSGYTFTTYTIHWVRQAPGKGLEWIGNINPETGG |
| heavy chain | | TTYNQKFEDKATLTVDKSTDTAYMELSSLRSEDTAVYYCAAGWNFDYWGQGTLLTVS |
| (HC) | | SASTKGPSVFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKSCDKTHTCPPCP |
| | | APELLGGPSVFLFPPKPKDTLMISRTPEVTCVVVDVSHEDPEVKENWYVDGVEVHNA |
| | | KTKPREEQYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |
| PSMA-Ab3 | 45 | DIQMTQSPSSLSTSVGDRVTLTCKASQDIGTAVDWYQQKPGKSPKLLIYWASTTHEG |
| light chain | | IPSRFSGSGSGTDFTLTISSLQPEDAADYYCQQYSSYPLTFGPGTKVEIKRTVAAPS |
| (LC) | | VFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSENRGEC |
| PSMA-Ab4 | 46 | EVQLVQSGPEVKKPGATVKISCKTSGYTFTTYTIHWVRQAPGKGLEWIGNINPQTGG |
| heavy chain | | TTYNQKFEDKATLTVDKSTDTAYMELSSLRSEDTAVYYCAAGWNFDYWGQGTLLTVS |
| variable | | S |
| region (VH) | | |
| PSMA-Ab4 | 47 | DIQMTQSPSSLSTSVGDRVTLTCKASQDIGTAVDWYQQKPGKSPKLLIYWASTTHEG |
| light chain | | IPSRFSGSGSGTDFTLTISSLQPEDAADYYCQQYSQYPLTFGPGTKVEIK |
| variable | | |
| region (VL) | | |
| PSMA-Ab4 | 48 | TYTIH |
| HCDR1 | | |
| (Kabat) | | |
| PSMA-Ab4 | 49 | NINPQTGGTTYNQKFED |
| HCDR2 | | |
| (Kabat) | | |
| PSMA-Ab4 | 50 | GWNFDY |
| HCDR3 | | |
| (Kabat) | | |
| PSMA-Ab4 | 51 | KASQDIGTAVD |
| LCDR1 | | |
| (Kabat) | | |
| PSMA-Ab4 | 52 | WASTTHE |
| LCDR2 | | |
| (Kabat) | | |
| PSMA-Ab4 | 53 | QQYSQYPLT |
| LCDR3 | | |
| (Kabat) | | |
| PSMA-Ab4 | 54 | EVQLVQSGPEVKKPGATVKISCKTSGYTFTTYTIHWVRQAPGKGLEWIGNINPQTGG |
| heavy chain | | TTYNQKFEDKATLTVDKSTDTAYMELSSLRSEDTAVYYCAAGWNFDYWGQGTLLTVS |
| (HC) | | SASTKGPSVFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKSCDKTHTCPPCP |
| | | APELLGGPSVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |
| PSMA-Ab4 | 55 | DIQMTQSPSSLSTSVGDRVTLTCKASQDIGTAVDWYQQKPGKSPKLLIYWASTTHEG |
| light chain | | IPSRFSGSGSGTDFTLTISSLQPEDAADYYCQQYSQYPLTFGPGTKVEIKRTVAAPS |
| (LC) | | VFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSENRGEC |
| PSMA-Ab5 | 56 | EVQLVQSGPEVKKPGATVKISCKTSGYTFTEYTIHWVRQAPGKGLEWIGNINPREGG |
| heavy chain | | TTYNQKFEDKATLTVDKSTDTAYMELSSLRSEDTAVYYCAAGWNFDYWGQGTLLTVS |
| variable | | S |
| region (VH) | | |
| PSMA-Ab5 | 57 | DIQMTQSPSSLSTSVGDRVTLTCKASQDIGTAVDWYQQKPGKSPKLLIYWASTRHEG |
| light chain | | IPSRFSGSGSGTDFTLTISSLQPEDAADYYCQQYSSYPLTFGPGTKVEIK |
| variable | | |
| region (VL) | | |
| PSMA-Ab5 | 58 | EYTIH |
| HCDR1 | | |
| (Kabat) | | |
| PSMA-Ab5 | 59 | NINPREGGTTYNQKFED |
| HCDR2 | | |
| (Kabat) | | |
| PSMA-Ab5 | 60 | GWNFDY |
| HCDR3 | | |
| (Kabat) | | |
| PSMA-Ab5 | 61 | KASQDIGTAVD |
| LCDR1 | | |
| (Kabat) | | |
| PSMA-Ab5 | 62 | WASTRHE |
| LCDR2 | | |
| (Kabat) | | |
| PSMA-Ab5 | 63 | QQYSSYPLT |
| LCDR3 | | |
| (Kabat) | | |
| PSMA-Ab5 | 64 | EVQLVQSGPEVKKPGATVKISCKTSGYTFTEYTIHWVRQAPGKGLEWIGNINPREGG |
| heavy chain | | TTYNQKFEDKATLTVDKSTDTAYMELSSLRSEDTAVYYCAAGWNFDYWGQGTLLTVS |
| (HC) | | SASTKGPSVFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKSCDKTHTCPPCP |
| | | APELLGGPSVFLFPPKPKDTLMISRTPEVTCVVVDVSHEDPEVKENWYVDGVEVHNA |
| | | KTKPREEQYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |
| PSMA-Ab5 | 65 | DIQMTQSPSSLSTSVGDRVTLTCKASQDIGTAVDWYQQKPGKSPKLLIYWASTRHEG |
| light chain | | IPSRFSGSGSGTDFTLTISSLQPEDAADYYCQQYSSYPLTFGPGTKVEIKRTVAAPS |
| (LC) | | VFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSENRGEC |

1. An isolated antibody or antigen-binding portion thereof that specifically binds to PSMA, wherein the antibody or antigen-binding portion thereof comprises:
(a) a heavy chain complementarity determining region 1 (HCDR1) comprising an amino acid sequence selected from SEQ ID NOs: 3-5; a heavy chain complementarity determining region 2 (HCDR2) comprising an amino acid sequence selected from SEQ ID NOs: 6-9; a heavy chain complementarity determining region 3 (HCDR3) comprising the amino acid sequence of SEQ ID NO: 10 or 11; a light chain complementarity determining region 1 (LCDR1) comprising the amino acid sequence of SEQ ID NO: 12 or 13; a light chain complementarity determining region 2 (LCDR2) comprising the amino acid sequence of SEQ ID NO: 14 or 15; and a light chain complementarity determining region 3 (LCDR3) comprising the amino acid sequence of SEQ ID NO: 16;
(b) HCDR1 comprising an amino acid sequence selected from SEQ ID NOs: 29-32: HCDR2 comprising an amino acid sequence selected from SEQ ID NOs: 33-36: HCDR3 comprising the amino acid sequence of SEQ ID NOs: 37 or 38: LCDR1 comprising the amino acid sequence of SEQ ID NO: 39 or 40: LCDR2 comprising the amino acid sequence of SEQ ID NO: 41 or 42; and LCDR3 comprising the amino acid sequence of SEQ ID NO: 43;
(c) HCDR1 comprising the amino acid sequence of SEQ ID NO: 48, HCDR2 comprising the amino acid sequence of SEQ ID NO: 49: HCDR3 comprising the amino acid sequence of SEQ ID NO: 50; LCDR1 comprising the amino acid sequence of SEQ ID NO: 51: LCDR2 comprising the amino acid sequence of SEQ ID NO: 52; and LCDR3 comprising the amino acid sequence of SEQ ID NO: 53; or
(d) HCDR1 comprising the amino acid sequence of SEQ ID NO: 58: HCDR2 comprising the amino acid sequence of SEQ ID NO: 59: HCDR3 comprising the amino acid sequence of SEQ ID NO: 60; LCDR1 comprising the amino acid sequence of SEQ ID NO: 61: LCDR2 comprising the amino acid sequence of SEQ ID NO: 62, and LCDR3 comprising the amino acid sequence of SEQ ID NO: 63.

2. The isolated antibody or antigen-binding portion thereof of claim 1, wherein the antibody or antigen-binding portion thereof comprises:
(a) HCDR1 comprising the amino acid sequence of SEQ ID NO: 3, HCDR2 comprising the amino acid sequence of SEQ ID NO: 6, HCDR3 comprising the amino acid sequence of SEQ ID NO: 10, LCDR1 comprising the amino acid sequence of SEQ ID NO: 12, LCDR2 comprising the amino acid sequence of SEQ ID NO: 14, and LCDR3 comprising the amino acid sequence of SEQ ID NO: 16;
(b) HCDR1 comprising the amino acid sequence of SEQ ID NO: 4, HCDR2 comprising the amino acid sequence of SEQ ID NO: 7, HCDR3 comprising the amino acid sequence of SEQ ID NO: 10, LCDR1 comprising the amino acid sequence of SEQ ID NO: 12, LCDR2 comprising the amino acid sequence of SEQ ID NO: 14, and LCDR3 comprising the amino acid sequence of SEQ ID NO: 16;
(c) HCDR1 comprising the amino acid sequence of SEQ ID NO: 4, HCDR2 comprising the amino acid sequence of SEQ ID NO: 8, HCDR3 comprising the amino acid sequence of SEQ ID NO: 11, LCDR1 comprising the amino acid sequence of SEQ ID NO: 13, LCDR2 comprising the amino acid sequence of SEQ ID NO: 15, and LCDR3 comprising the amino acid sequence of SEQ ID NO: 16;
(d) HCDR1 comprising the amino acid sequence of SEQ ID NO: 5, HCDR2 comprising the amino acid sequence of SEQ ID NO: 9, HCDR3 comprising the amino acid sequence of SEQ ID NO: 10, LCDR1 comprising the amino acid sequence of SEQ ID NO: 12, LCDR2 comprising the amino acid sequence of SEQ ID NO: 14, and LCDR3 comprising the amino acid sequence of SEQ ID NO: 16;
(e) HCDR1 comprising the amino acid sequence of SEQ ID NO:29, HCDR2 comprising the amino acid sequence of SEQ ID NO:33, HCDR3 comprising the amino acid sequence of SEQ ID NO:37, LCDR1 comprising the amino acid sequence of SEQ ID NO:39, LCDR2 comprising the amino acid sequence of SEQ ID NO:41, and LCDR3 comprising the amino acid sequence of SEQ ID NO:43;
(f) HCDR1 comprising the amino acid sequence of SEQ ID NO:30, HCDR2 comprising the amino acid sequence of SEQ ID NO:34, HCDR3 comprising the amino acid sequence of SEQ ID NO:37, LCDR1 comprising the amino acid sequence of SEQ ID NO:39, LCDR2 comprising the amino acid sequence of SEQ ID NO:41, and LCDR3 comprising the amino acid sequence of SEQ ID NO:43;
(g) HCDR1 comprising the amino acid sequence of SEQ ID NO: 31, HCDR2 comprising the amino acid sequence of SEQ ID NO: 35, HCDR3 comprising the amino acid sequence of SEQ ID NO: 38, LCDR1 comprising the amino acid sequence of SEQ ID NO:40, LCDR2 comprising the amino acid sequence of SEQ ID NO:42, and LCDR3 comprising the amino acid sequence of SEQ ID NO:43: or
(h) HCDR1 comprising the amino acid sequence of SEQ ID NO: 32, HCDR2 comprising the amino acid sequence of SEQ ID NO:36, HCDR3 comprising the amino acid sequence of SEQ ID NO:37, LCDR1 comprising the amino acid sequence of SEQ ID NO:39, LCDR2 comprising the amino acid sequence of SEQ ID NO:41, and LCDR3 comprising the amino acid sequence of SEQ ID NO:43.

3. The isolated antibody or antigen-binding portion thereof of claim 1, wherein the antibody or antigen-binding portion thereof comprises a heavy chain variable region (VH) comprising an amino acid sequence that is at least 95% identical to the amino acid sequence of SEQ ID NO:1, 27, 46, or 56, and a light chain variable region (VL) comprising an amino acid sequence that is at least 95% identical to the amino acid sequence of SEQ ID NO: 2, 28, 47, or 57; optionally wherein the glutamic acid (E) at the N-terminus of SEQ ID NO: 1, 27, 46, or 56 is replaced with a pyroglutamic acid (pE).

4. The isolated antibody or antigen-binding portion thereof of claim 1, wherein the antibody or antigen-binding portion thereof comprises a heavy chain variable region (VH) comprising the amino acid sequence of SEQ ID NO: 1, 27, 46, or 56, and a light chain variable region (VL) comprising the amino acid sequence selected of SEQ ID NO: 2, 28, 47, or 57; optionally wherein the glutamic acid (E) at the N-terminus of SEQ ID NO: 1, 27, 46, or 56 is replaced with a pyroglutamic acid (pE).

5. (canceled)

6. (canceled)

7. The isolated antibody or antigen-binding portion thereof of claim 1, wherein the antibody or antigen-binding portion thereof is an IgG antibody.

8. The isolated antibody or antigen-binding portion thereof of claim 7, wherein the antibody is an IgG antibody lacking a C-terminal lysine in the heavy chain constant region.

9. The isolated antibody or antigen-binding portion thereof of claim 7, wherein the antibody is an IgG1 antibody, an IgG2 antibody, an IgG3 antibody, or an IgG4 antibody.

10. The isolated antibody or antigen-binding portion thereof of claim 1, wherein the antibody is a full-length antibody.

11. The isolated antibody or antigen-binding portion thereof of claim 1, wherein the antibody or antigen-binding portion thereof comprises a heavy chain (HC) comprising an amino acid sequence that is at least 95% identical to the amino acid sequence of SEQ ID NO: 17, 44, 54, or 64, and a light chain (LC) comprising an amino acid sequence that is at least 95% identical to the amino acid sequence of SEQ ID NO: 18, 45, 55, 65; optionally wherein the glutamic acid (E) at the N-terminus of SEQ ID NO: 17, 44, 54, or 64, is replaced with a pyroglutamic acid (pE).

12. The isolated antibody or antigen-binding portion thereof of claim 1, wherein the antibody or antigen-binding portion thereof comprises a heavy chain (HC) comprising the amino acid sequence of SEQ ID NO: 17, and a light chain (LC) comprising the amino acid sequence selected of SEQ ID NO: 18; optionally wherein the glutamic acid (E) at the N-terminus of SEQ ID NO: 17 is replaced with a pyroglutamic acid (pE).

13. The isolated antibody or antigen-binding portion thereof of claim 1, wherein the antibody or antigen-binding portion thereof is an antibody fragment selected from an Fv, single-chain Fv (scFv), Fab, Fab′, or (Fab′)₂.

14. (canceled)

15. (canceled)

16. The isolated antibody or antigen-binding portion thereof of claim 1, wherein the antibody or antigen-binding portion thereof binds to human PSMA comprising the amino acid sequence of SEQ ID NO: 21 or SEQ ID NO: 26 with an affinity (K_{D}) of less than 10 nM or less than 5 nM, as determined using surface plasmon resonance.

17. The isolated antibody or antigen-binding portion thereof of claim 1, wherein the antibody or antigen-binding portion thereof binds to cynomolgus monkey PSMA with an affinity (K_{D}) of less than 10 nM or less than 7 nM, as determined using surface plasmon resonance.

18. (canceled)

19. The isolated antibody or antigen-binding portion thereof of claim 1, wherein the antibody or antigen-binding portion thereof is a bispecific antibody.

20. An antibody-drug conjugate comprising the antibody or antigen-binding portion thereof claim 1 and a cytotoxic agent.

21. A pharmaceutical composition comprising the antibody or antigen-binding portion thereof of claim 1 and a pharmaceutically acceptable carrier.

22. An isolated nucleic acid encoding the antibody or antigen-binding portion thereof of claim 1.

23. (canceled)

24. (canceled)

25. (canceled)

26. (canceled)

27. (canceled)

28. A method of treating cancer in a human subject in need thereof, comprising administering to the subject a therapeutically effective amount of the isolated antibody or antigen-binding portion thereof of claim 1.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. An isolated antibody or antigen-binding portion thereof that specifically binds to PSMA, wherein the antibody or antigen-binding portion thereof comprises a heavy chain variable region (VH) comprising a heavy chain complementarity determining region 1 (HCDR1) comprising the amino acid sequence of SEQ ID NOs: 3; a heavy chain complementarity determining region 2 (HCDR2) comprising the amino acid sequence of SEQ ID NOs: 6; and a heavy chain complementarity determining region 3 (HCDR3) comprising the amino acid sequence of SEQ ID NO: 10; and a light chain variable region (VL) comprising a light chain complementarity determining region 1 (LCDR1) comprising the amino acid sequence of SEQ ID NO: 12; a light chain complementarity determining region 2 (LCDR2) comprising the amino acid sequence of SEQ ID NO: 14; and a light chain complementarity determining region 3 (LCDR3) comprising the amino acid sequence of SEQ ID NO: 16.

41. The isolated antibody or antigen-binding portion thereof of claim 40, wherein the VH comprises the amino acid sequence of SEQ ID NO: 1, optionally wherein the glutamic acid (E) at the N-terminus of SEQ ID NO: 1 is replaced with a pyroglutamic acid (pE); and wherein the VL comprises the amino acid sequence of SEQ ID NO: 2.

42. An antibody-drug conjugate comprising the antibody or antigen-binding portion thereof of claim 41.

43. An antibody-drug conjugate comprising the antibody or antigen-binding portion thereof of claim 12.
